SEQUENCE LISTING (46 sequences)

SEQ ID NO: 1 - protein, 238 aa, synthetic construct
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 2 - protein, 122 aa, synthetic construct
QVTLKESGPGILQPSQTLSLTCSFSGFSLNTSAMGVGWIRQPSGKGLEWLAHIWWNDDHYYNPALKSRVTISKDASNNQVFLKIASVVTADTATYYCARSIAPVVPTPFAYWGQGTLVTVSA

SEQ ID NO: 3 - DNA, 366 bp, synthetic construct
  source 1..366: mol_type = other DNA
caggttactctgaaagagtctggccctgggatattgcagccctcccagaccctcagtctgacttgttctttttctgggttttcactgaacacttctgctatgggtgtaggctggattcgtcagccttcagggaagggtctggagtggctggcacacatttggtggaatgatgatcactactataatccagccctgaaaagccgggtcacaatctccaaggatgcctccaacaaccaggtattcctcaagatcgccagtgtggtcactgcagatactgccacatactactgtgctcgatctattgctccggtagtgcctacaccgtttgcttactggggccaagggactctggtcactgtctctgca

SEQ ID NO: 4 - protein, 107 aa, synthetic construct
DIQMTQSSSSFSVSLGDRVTITCKASEDIYNRLAWFQQKPGNAPRLLISGATRLETGVPSRFSGSGSGKDYTLSITSLQTEDVGTYYCQQYWSSSWTFGGGTKLEIK

SEQ ID NO: 5 - DNA, 321 bp, synthetic construct
  source 1..321: mol_type = other DNA
gacatccagatgacacaatcttcatcctccttttctgtatctctaggagacagagtcaccattacttgcaaggcaagtgaggacatatataatcggttagcctggtttcagcagaaaccaggaaatgctcctaggctcttaatatctggtgcaaccagattggaaactggggttccttcaagattcagtggcagtggatctggaaaggattacactctcagcattaccagtcttcagactgaagatgttggtacttattactgtcaacagtattggagttcttcgtggacgttcggtggaggcaccaagctggaaatcaaa

SEQ ID NO: 6 - protein, 7 aa, synthetic construct
TSAMGVG

SEQ ID NO: 7 - protein, 9 aa, synthetic construct
GFSLNTSAM

SEQ ID NO: 8 - protein, 12 aa, synthetic construct
GFSLNTSAMGVG

SEQ ID NO: 9 - protein, 16 aa, synthetic construct
HIWWNDDHYYNPALKS

SEQ ID NO: 10 - protein, 5 aa, synthetic construct
WWNDD

SEQ ID NO: 11 - protein, 12 aa, synthetic construct
SIAPVVPTPFAY

SEQ ID NO: 12 - protein, 11 aa, synthetic construct
KASEDIYNRLA

SEQ ID NO: 13 - protein, 7 aa, synthetic construct
GATRLET

SEQ ID NO: 14 - protein, 9 aa, synthetic construct
QQYWSSSWT

SEQ ID NO: 15 - protein, 115 aa, synthetic construct
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDIWGQGTLVTVSS

SEQ ID NO: 16 - protein, 108 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 17 - protein, 115 aa, synthetic construct
EVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSS

SEQ ID NO: 18 - protein, 108 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIK

SEQ ID NO: 19 - protein, 237 aa, synthetic construct
EVQLLESGGGVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIK

SEQ ID NO: 20 - protein, 122 aa, synthetic construct
EVQLQESGPGILQPSQTLSLTCSFSGFSLSTSGMNVGWIRQPSGKGLEWLAHMWNDDNYYNPALKSRLTISKDPSNNQVFLKIASVVTADTATYFCARLEESTTITTAFAYWGQGTLVTVSA

SEQ ID NO: 21 - DNA, 367 bp, synthetic construct
  source 1..367: mol_type = other DNA
gaggtgcagctgcaggagtctggccctgggatattgcagccctcccagaccctcagtctgacttgttctttctctgggttttcactgagcacttctggtatgaatgtaggctggattcgtcagccttcagggaagggtctggagtggctggcacacatgtggaatgatgataactactataacccagccctgaaaagccggctcacaatctccaaggatccctccaacaaccaggtattcctcaagatcgccagtgtggtcactgcagatactgccacatacttctgtgctcgattagaggaatctactacgattacgacggcttttgcttattggggccaagggactctggtcactgtctccgcag

SEQ ID NO: 22 - protein, 108 aa, synthetic construct
EIVVTQSPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLEIK

SEQ ID NO: 23 - DNA, 325 bp, synthetic construct
  source 1..325: mol_type = other DNA
gaaattgtggtcacccagtctccagcactcatggctgcatctccgggggagaaggtcaccatcacctgcagtgtcagctctagtataagttccagcaatttgcactggtaccagcagaagtcagaaacctcccccaaaccctggatttatggcacatccaacctggcttctggagtccctgttcgcttcagtggcagtggatctgggacctcttattctctcacaatcagcagcatggaggctgaagatgctgccacttattattgtcaacagtggagtagttacccgctcacgttcggtgctgggaccaagctggaaataaaac

SEQ ID NO: 24 - protein, 7 aa, synthetic construct
TSGMNVG

SEQ ID NO: 25 - protein, 9 aa, synthetic construct
GFSLSTSGM

SEQ ID NO: 26 - protein, 12 aa, synthetic construct
GFSLSTSGMNVG

SEQ ID NO: 27 - protein, 15 aa, synthetic construct
HMWNDDNYYNPALKS

SEQ ID NO: 28 - protein, 4 aa, synthetic construct
WNDD

SEQ ID NO: 29 - protein, 13 aa, synthetic construct
LEESTTITTAFAY

SEQ ID NO: 30 - protein, 12 aa, synthetic construct
SVSSSISSSNLH

SEQ ID NO: 31 - protein, 7 aa, synthetic construct
GTSNLAS

SEQ ID NO: 32 - protein, 9 aa, synthetic construct
QQWSSYPLT

SEQ ID NO: 33 - protein, 117 aa, synthetic construct
QVQLQQSGPGLVQPSQSLSIPCTVSGFSLTSYDVHWVRQSPGKGLEWLGVIWSGGSTGYNAIFRSRLSINKDNSKSQVFFKMTSLQPDDTAIYYCANFSPWYLPSYWDQGTLVTVSA

SEQ ID NO: 34 - DNA, 352 bp, synthetic construct
  source 1..352: mol_type = other DNA
caggtgcagctgcagcagtcaggacctggcctagtgcagccctcacagagcctgtccatcccctgcacagtctctggtttctcattaactagctatgatgtacattgggttcgccagtctccaggaaagggtctggagtggctgggagtgatctggagtggtggcagcacaggctataatgcaattttcagatccagactgagcatcaacaaggacaactccaagagccaagttttctttaaaatgaccagtctgcaacctgatgacacagccatatattactgtgccaatttctccccgtggtacctcccttcttactgggaccaagggactctggtcactgtctctgcag

SEQ ID NO: 35 - protein, 106 aa, synthetic construct
QIVLTQSPAIMSASPGEKVTITCSASSNVSYMHWFQQKPGTSPKLWIYSTSNLASGVPARFSGSGSGTSYSLTISRMEAEDAATYYCQQRSSYPLTFGAGTKLELK

SEQ ID NO: 36 - DNA, 319 bp, synthetic construct
  source 1..319: mol_type = other DNA
caaattgttctcacccagtctccagcaatcatgtctgcttctccaggggagaaggtcaccataacctgcagtgccagctcaaatgtcagttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagtagttacccactcacgttcggtgctgggactaagctggagctgaaac

SEQ ID NO: 37 - protein, 5 aa, synthetic construct
SYDVH

SEQ ID NO: 38 - protein, 7 aa, synthetic construct
GFSLTSY

SEQ ID NO: 39 - protein, 10 aa, synthetic construct
GFSLTSYDVH

SEQ ID NO: 40 - protein, 16 aa, synthetic construct
VIWSGGSTGYNAIFRS

SEQ ID NO: 41 - protein, 5 aa, synthetic construct
WSGGS

SEQ ID NO: 42 - protein, 9 aa, synthetic construct
FSPWYLPSY

SEQ ID NO: 43 - protein, 10 aa, synthetic construct
SASSNVSYMH

SEQ ID NO: 44 - protein, 7 aa, synthetic construct
STSNLAS

SEQ ID NO: 45 - protein, 9 aa, synthetic construct
QQRSSYPLT

SEQ ID NO: 46 - protein, 8 aa, synthetic construct
HHHHHHHH

(19) United States
(12) Patent Application Publication
(10) Pub. No.: US 2024/0101710 A1
(43) Pub. Date: Mar. 28, 2024

(54) B-CELL MATURATION ANTIGEN (BCMA) ANTI-IDIOTYPIC ANTIBODIES

(71) Applicants: ALLOGENE THERAPEUTICS, INC., South San Francisco, CA (US); PFIZER INC., New York, NY (US)

(72) Inventors: Zea Melton, Pacifica, CA (US); Tao Sai, Foster City, CA (US); Thomas John Van Blarcom, Oakland, CA (US)

(21) Appl. No.: 18/468,854

(22) Filed: Sep. 18, 2023

Related U.S. Application Data
(60) Provisional application No. 63/376,156, filed on Sep. 19, 2022.

(51) Int. Cl. C07K 16/42; A61K 49/00; C07K 1/22; C07K 16/28; C12N 5/0783; G01N 33/569

(52) U.S. Cl. CPC C07K 16/4266; A61K 49/0058; C07K 1/22; C07K 16/2878; C12N 5/0636; G01N 33/56972; C07K 2317/622; C12N 2501/998

(57) ABSTRACT

Provided herein are anti-idiotype antibodies that specifically recognize anti-BCMA antibody moieties, in particular, anti-BCMA antibody moieties present in recombinant receptors, including chimeric antigen receptors (CARs). The disclosure further relates to uses of anti-idiotype antibodies for specifically identifying and/or selecting cells expressing such recombinant receptors, such as anti-BCMA CAR T cells. The disclosure further relates to uses of anti-idiotype antibodies for specifically activating such cells.

### CROSS REFERENCE TO RELATED APPLICATIONS

The present application claims the benefit of priority to U.S. Provisional Application No. 63/376,156, filed on Sep. 19, 2022, the content of which is hereby incorporated by reference in its entirety.

### REFERENCE TO SEQUENCE LISTING

The instant application contains a Sequence Listing which has been submitted electronically in XML file format and is hereby incorporated by reference in its entirety. Said XML copy, created on Aug. 31, 2023, is named AT-036_02WO_SL.xml and is 44,155 bytes in size.

### TECHNICAL FIELD

The present disclosure relates in some aspects to anti-idiotype (anti-id) antibodies that specifically recognize anti-BCMA antibody moieties, in particular, anti-BCMA antibody moieties present in recombinant receptors, including chimeric antigen receptors (CARs). The disclosure further relates to uses of anti-idiotype antibodies, e.g., for specifically identifying, quantifying or selecting cells expressing such recombinant receptors, such as anti-BCMA CAR T cells. The disclosure further relates to uses of anti-idiotype antibodies for specifically activating such cells.

### BACKGROUND

Anti-idiotypic antibodies are a subset of antibodies raised against immunizing antibodies. These anti-idiotypic antibodies demonstrated specific binding against the idiotopes (unique antigenic determinants on the surface of the antibodies) of the immunizing antibodies. Anti-idiotypic antibodies can be generally classified into three distinct groups: (1) antibodies that recognize idiotopes distinct from the antigen-binding site (ABS) on immunizing antibodies; (2) antibodies that recognize epitopes within the ABS and mimic the structure of the nominal antigen; and (3) antibodies that recognize epitopes within the ABS without the structural resemblance of the nominal antigen (see, e.g., Pan et al., (1995) FASEB J 9:43-49).

Conventional methods for detecting CAR expression, such as anti-BCMA CAR expression (e.g., using soluble human BCMA-Fc fusion protein) can lack specificity, have batch-to-batch variability, produce low intensity signals in analytical methods (e.g., flow cytometry), or be difficult to make in sufficient quantities, which can underestimate the true number CAR expressing cells.

Thus, there is a need for robust reagents to accurately detect anti-BCMA CAR expression on engineered T cells or in in vitro assays. Provided herein are methods and compositions addressing this and other needs.

### SUMMARY

Provided herein are agents that specifically bind to antibodies, including antibody fragments such as single chain variable fragments (scFvs), and chimeric molecules containing the same, such as chimeric antigen receptors. Also among the embodiments provided herein are uses and methods of using such agents, including for detection, use, manipulation, and/or stimulation of cells or therapies containing or suspected of containing the antibody or chimeric molecule, such as in the detection, stimulation or use of CAR-expressing cells.

In some aspects, the antibody is or includes an anti-idiotype (anti-id) antibody or antigen-binding fragment thereof that specifically binds to a target antibody that is or contains variable region(s) of the antibody designated C29, and/or specifically binds to a chimeric molecule containing such an antibody fragment, such as a CAR with a binding domain containing antibody variable regions or portions thereof derived from C29, such as in the form of an scFv. In some aspects, the antibody is or includes an anti-idiotype antibody or antigen-binding fragment thereof that specifically binds to a target antibody that is or contains variable region(s) of the antibody designated P5A2, which exhibits high sequence identity to C29 in the heavy and light chain variable regions, and/or specifically binds to a chimeric molecule containing such an antibody fragment, such as a CAR with a binding domain containing antibody variable regions or portions thereof derived from P5A2, such as in the form of an scFv.

In some embodiments, the anti-BCMA scFv derived from C29 comprises an amino acid sequence that is at least 80%, at least 90%, at least 94%, at least 95%, at least 96%, at least 97%, at least 98% at least 99%, or 100% sequence identity with the amino acid sequence of SEQ ID NO: 1. In some embodiments, the anti-BCMA scFv derived from P5A2 comprises an amino acid sequence that is at least 80%, at least 90%, at least 94%, at least 95%, at least 96%, at least 97%, at least 98%, at least 99%, or 100% sequence identity with the amino acid sequence of SEQ ID NO: 19. In some embodiments, the isolated antibody does not bind to a framework region of C29 or P5A2. In some embodiments, the isolated antibody binds to an anti-BCMA scFv derived from C29 or P5A2 with a KD of no more than 10 nM, no more than 5 nM, no more than 1 nM, no more than 100 pM, no more than 90 pM, no more than 80 pM, no more than 70 pM, no more than 60 pM, no more than 50 pM, no more than 40 pM, no more than 30 pM, or no more than 20 pM, as determined by a Biacore assay at 25° C. In various embodiments an antigen binding molecule is selected from the group consisting of an antibody, an scFv, a Fab, a Fab′, a Fv, a F(ab′)₂, a dAb, a human antibody, a humanized antibody, a chimeric antibody, a monoclonal antibody, a polyclonal antibody, a recombinant antibody, an IgE antibody, an IgD antibody, an IgM antibody, an IgG1 antibody, an IgG1 antibody having at least one mutation in the hinge region, an IgG2 antibody an IgG2 antibody having at least one mutation in the hinge region, an IgG3 antibody, an IgG3 antibody having at least one mutation in the hinge region, an IgG4 antibody, an IgG4 antibody having at least one mutation in the hinge region, an antibody comprising at least one non-naturally occurring amino acid, and any combination thereof.

In further embodiments, an isolated anti-idiotypic antibody comprises a heavy chain (HC), and in specific embodiments the HC comprises a heavy chain variable region (VH) amino acid sequence of SEQ ID NO: 2. In further specific embodiments of an antibody provided herein comprises a heavy chain CDR1 selected from the group consisting of SEQ ID NOs: 6, 7, and 8. In additional specific embodiments of an antibody provided herein comprises a heavy chain CDR2 selected from the group consisting of SEQ ID NOs: 9 and 10. In yet other embodiments of an antibody provided herein comprises a heavy chain CDR3 selected from the group consisting of SEQ ID NO: 11. In still further embodiments, a heavy chain of an antibody provided herein comprises a heavy chain CDR1, a heavy chain CDR2, and a heavy chain CDR3, each CDR comprising an amino acid sequence shown in Table 1c.

In some embodiments, an anti-idiotypic antibody comprises a VH amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule provided herein.

In some embodiments, an isolated anti-idiotypic antibody provided herein comprises a light chain (LC), and in various embodiments a LC comprises a light chain variable region (VL) sequence of SEQ ID NO: 4. In some embodiments a light chain variable region (VL) of an antibody provided herein comprises one or more of (a) a CDR1, (b) a CDR2, and (c) a CDR3. In further specific embodiments, a light chain CDR1 of an antibody provided herein can comprise SEQ ID NO: 12. In other embodiments, a light chain CDR2 of an antibody provided herein can comprise SEQ ID NO: 13. In still further embodiments a light chain CDR3 of an antibody provided herein can comprise SEQ ID NO: 14. In still further embodiments, a light chain of an antibody provided herein comprises a light chain CDR1, a light chain CDR2 and a light chain CDR3, each CDR comprising an amino acid sequence in Table 1d.

In some embodiments, a VL amino acid sequence that is at least about 70%, at least about 75%, at least about 80%, at least about 85%, at least about 90%, at least about 95%, at least about 96%, at least about 97%, at least about 98%, at least about 99%, or about 100% identical to a VL of an antigen binding molecule are provided herein.

In some embodiments, an anti-idiotypic antibody provided herein comprises (a) a VH comprising the amino acid sequence of SEQ ID NO: 2; and (b) a VL comprising the amino acid sequence of SEQ ID NO: 4. In another embodiment, an antibody provided herein comprises (a) a VH CDR1 comprising the amino acid sequence of SEQ ID NO:6, 7 or 8; (b) a VH CDR2 comprising the amino acid sequence of SEQ ID NO: 9 or 10; (c) a VH CDR3 comprising the amino acid sequence of SEQ ID NO: 11; (d) a VL CDR1 comprising the amino acid sequence of SEQ ID NO: 12; (e) a VL CDR2 comprising the amino acid sequence of SEQ ID NO: 13; and (f) a VL CDR3 comprising the amino acid sequence of SEQ ID NO: 14.

In further embodiments, an isolated anti-idiotypic antibody comprises a heavy chain (HC), and in specific embodiments the HC comprises a heavy chain variable region (VH) amino acid sequence of SEQ ID NO: 20. In further specific embodiments of an antibody provided herein comprises a heavy chain CDR1 selected from the group consisting of SEQ ID NOs: 24, 25 and 26. In additional specific embodiments of an antibody provided herein comprises a heavy chain CDR2 selected from the group consisting of SEQ ID NOs: 27 and 28. In yet other embodiments of an antibody provided herein comprises a heavy chain CDR3 selected from the group consisting of SEQ ID NO: 29. In still further embodiments, a heavy chain of an antibody provided herein comprises a heavy chain CDR1, a heavy chain CDR2, and a heavy chain CDR3, each CDR comprising an amino acid sequence shown in Table 1c.

In some embodiments, an anti-idiotypic antibody comprises a VH amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule provided herein.

In some embodiments, an isolated anti-idiotypic antibody provided herein comprises a light chain (LC), and in various embodiments a LC comprises a light chain variable region (VL) sequence of SEQ ID NO: 22. In some embodiments a light chain variable region (VL) of an antibody provided herein comprises one or more of (a) a CDR1, (b) a CDR2, and (c) a CDR3. In further specific embodiments, a light chain CDR1 of an antibody provided herein can comprise SEQ ID NO: 30. In other embodiments, a light chain CDR2 of an antibody provided herein can comprise SEQ ID NO: 31. In still further embodiments a light chain CDR3 of an antibody provided herein can comprise SEQ ID NO: 32. In still further embodiments, a light chain of an antibody provided herein comprises a light chain CDR1, a light chain CDR2 and a light chain CDR3, each CDR comprising an amino acid sequence in Table 1d.

In some embodiments, a VL amino acid sequence that is at least about 70%, at least about 75%, at least about 80%, at least about 85%, at least about 90%, at least about 95%, at least about 96%, at least about 97%, at least about 98%, at least about 99%, or about 100% identical to a VL of an antigen binding molecule are provided herein.

In some embodiments, an anti-idiotypic antibody provided herein comprises (a) a VH comprising the amino acid sequence of SEQ ID NO: 20; and (b) a VL comprising the amino acid sequence of SEQ ID NO: 22. In another embodiment, an antibody provided herein comprises (a) a VH CDR1 comprising the amino acid sequence of SEQ ID NO: 24, 25 or 26; (b) a VH CDR2 comprising the amino acid sequence of SEQ ID NO: 27 or 28; (c) a VH CDR3 comprising the amino acid sequence of SEQ ID NO: 29; (d) a VL CDR1 comprising the amino acid sequence of SEQ ID NO: 30; (e) a VL CDR2 comprising the amino acid sequence of SEQ ID NO: 31; and (f) a VL CDR3 comprising the amino acid sequence of SEQ ID NO: 32.

In further embodiments, an isolated anti-idiotypic antibody comprises a heavy chain (HC), and in specific embodiments the HC comprises a heavy chain variable region (VH) amino acid sequence of SEQ ID NO: 33. In further specific embodiments of an antibody provided herein comprises a heavy chain CDR1 selected from the group consisting of SEQ ID NOs: 37, 38 and 39. In additional specific embodiments of an antibody provided herein comprises a heavy chain CDR2 selected from the group consisting of SEQ ID NOs: 40 and 41. In yet other embodiments of an antibody provided herein comprises a heavy chain CDR3 selected from the group consisting of SEQ ID NO: 42. In still further embodiments, a heavy chain of an antibody provided herein comprises a heavy chain CDR1, a heavy chain CDR2, and a heavy chain CDR3, each CDR comprising an amino acid sequence shown in Table 1c.

In some embodiments, an anti-idiotypic antibody comprises a VH amino acid sequence that is at least about 70%, at least about 75%, at least about 80%, at least about 85%, at least about 90%, at least about 95%, at least about 96%, at least about 97%, at least about 98%, at least about 99%, or about 100% identical to a VH of an antigen binding molecule provided herein.

In some embodiments, an isolated anti-idiotypic antibody provided herein comprises a light chain (LC), and in various embodiments a LC comprises a light chain variable region (VL) sequence of SEQ ID NO: 35. In some embodiments a light chain variable region (VL) of an antibody provided herein comprises one or more of (a) a CDR1, (b) a CDR2, and (c) a CDR3. In further specific embodiments, a light chain CDR1 of an antibody provided herein can comprise SEQ ID NO: 43. In other embodiments, a light chain CDR2 of an antibody provided herein can comprise SEQ ID NO: 44. In still further embodiments a light chain CDR3 of an antibody provided herein can comprise SEQ ID NO: 45. In still further embodiments, a light chain of an antibody provided herein comprises a light chain CDR1, a light chain CDR2 and a light chain CDR3, each CDR comprising an amino acid sequence in Table 1d.

In some embodiments, a VL amino acid sequence that is at least about 70%, at least about 75%, at least about 80%, at least about 85%, at least about 90%, at least about 95%, at least about 96%, at least about 97%, at least about 98%, at least about 99%, or about 100% identical to a VL of an antigen binding molecule are provided herein.

In some embodiments, an anti-idiotypic antibody provided herein comprises (a) a VH comprising the amino acid sequence of SEQ ID NO: 33; and (b) a VL comprising the amino acid sequence of SEQ ID NO: 35. In another embodiment, an antibody provided herein comprises (a) a VH CDR1 comprising the amino acid sequence of SEQ ID NO: 37, 38 or 39; (b) a VH CDR2 comprising the amino acid sequence of SEQ ID NO: 40 or 41; (c) a VH CDR3 comprising the amino acid sequence of SEQ ID NO: 42; (d) a VL CDR1 comprising the amino acid sequence of SEQ ID NO: 43; (e) a VL CDR2 comprising the amino acid sequence of SEQ ID NO: 44; and (f) a VL CDR3 comprising the amino acid sequence of SEQ ID NO: 45.

In some of any such embodiments, the target antibody or antigen-binding fragment is a single chain fragment. In some aspects, the fragment contains antibody variable regions joined by a flexible linker. In some of any such embodiments, the fragment contains an scFv.

In some of any such embodiments, the anti-idiotype antibody or antigen-binding fragment specifically binds to the same or an overlapping epitope of a target antibody or antigen-binding fragment thereof as the epitope specifically bound by the anti-id antibody or antigen-binding fragment according to any one of the embodiments described herein.

In some of any such embodiments, the target antibody or antigen-binding fragment is within or including in the antigen-binding domain of the extracellular portion of a chimeric antigen receptor; and/or the anti-id antibody or antigen-binding fragment specifically binds the target antibody or antigen-binding fragment contained within or included in the antigen-binding domain of the extracellular portion of a CAR. In some embodiments, the target antibody or antigen-binding fragment is an scFv and the anti-id antibody or antigen-binding fragment specifically binds to an epitope in the scFv of the CAR.

In some of any such embodiments, the antibody or fragment specifically binds to a scFv derived from antibody C29 contained in the extracellular portion of a chimeric antigen receptor, optionally wherein the scFv derived from antibody C29 contains a heavy chain variable region set forth in SEQ ID NO: 15 and/or a light chain variable region set forth in SEQ ID NO: 16 and/or contains the sequence of amino acids set forth in SEQ ID NO: 1. In some of any such embodiments, the antibody or fragment specifically binds to a scFv derived from antibody P5A2 comprised in the extracellular portion of a chimeric antigen receptor, optionally wherein the scFv derived from antibody P5A2 contains a heavy chain variable region set forth in SEQ ID NO: 17 and/or a light chain variable region set forth in SEQ ID NO: 18; and/or contains the sequence of amino acids set forth in SEQ ID NO: 19.

In some of any such embodiments, the anti-idiotype antibody or antigen-binding fragment specifically binds to an epitope within or including all or a portion of a complementarity determining region (CDR) of the target antibody or antigen-binding fragment.

In some of any such embodiments, the anti-idiotype antibody or fragment is antagonist antibody of a CAR containing the target antibody or antigen-binding fragment. In some of any such embodiments, the antibody or fragment is an antagonist of a CAR containing the target antibody or antigen-binding fragment.

In some of any such embodiments, the anti-idiotype antibody or antigen-binding fragment thereof is humanized. In some of any such embodiments, the anti-idiotype antibody or antigen-binding fragment thereof is recombinant. In some of any such embodiments, the anti-idiotype antibody or antigen-binding fragment thereof is monoclonal.

In some of any such embodiments, the anti-idiotype antibody or antigen-binding fragment thereof is an antigen-binding fragment. In some aspects, the antigen-binding fragment is selected from among fragment antigen binding (Fab) fragments, F(ab′) 2 fragments, Fab′ fragments, Fv fragments, a single chain variable fragment (scFv) or a single domain antibody.

In some of any such embodiments, the anti-idiotype antibody or antigen-binding fragment thereof contains at least a portion of an immunoglobulin constant region. In some embodiments, the at least a portion of an immunoglobulin constant region contains an Fc region or a portion of the Fc containing the CH2 and CH3 domains. In some aspects, the constant region is derived from human IgG. In some of any such embodiments, the anti-idiotype antibody or antigen-binding fragment is an intact antibody or full-length antibody.

Provided herein is a vector containing the nucleic acid molecule according to any one of the embodiments described herein. Also provided herein is a cell containing the anti-idiotype antibody or antigen-binding fragment thereof according to any one of the embodiments described herein or the nucleic acid molecule according to any one of the embodiments described herein.

Provided herein is a method of producing an anti-idiotype antibody or antigen-binding fragment thereof, including expressing the heavy and/or light chain encoded by the

nucleic acid molecule according to any one of the embodiments described herein or the vector according to any one of the embodiments described herein in a suitable host cell and recovering or isolating the antibody. In some embodiments, the method of producing an anti-idiotype antibody or antigen-binding fragment includes culturing the cell according to any one of the embodiments described herein under conditions in which the heavy chain and/or light chain is expressed and recovering or isolating the antibody. Also provided herein is an anti-idiotype antibody or antigen-binding fragment thereof produced by the method according to any one of the embodiments described herein.

Also provided are methods of detection using any of the provided agents, such as the anti-idiotype antibodies. In some embodiments, provided is a method of detecting a target antibody or antigen-binding fragment thereof, such as a CAR containing the same, including (a) contacting a composition containing a target antibody (such as one with variable regions derived from an antibody C29 or from an antibody P5A2, or from an antigen-binding fragment of either of such antibodies) with the anti-idiotype antibody or antigen-binding fragment thereof, and (b) detecting the anti-idiotype antibody bound to the target antibody or antigen-binding fragment and/or detecting the presence or absence of the target antibody or agent.

In some embodiments, the provided methods involve detecting a CAR containing a target antibody or antigen-binding fragment thereof of any of the embodiments, such as a CAR containing variable domains derived from C29 or P5A2. In some aspects, the methods include (a) contacting a cell expressing a chimeric antigen receptor (CAR) containing a target antibody that is the antibody C29 or an antigen-binding fragment thereof with the anti-idiotype antibody or antigen-binding fragment thereof according to any one of the embodiments described or the conjugate according to any one of the embodiments described that specifically binds to a target antibody that is antibody C29 or an antigen-binding fragment thereof, and (b) detecting cells bound with the anti-idiotype antibody. In some embodiments, the method includes (a) contacting a cell expressing a chimeric antigen receptor (CAR) containing a target antibody that is the antibody P5A2 or an antigen-binding fragment thereof with the anti-idiotype antibody or antigen-binding fragment thereof of any one of the embodiments described or the conjugate of any one of the embodiments described that specifically binds to a target antibody that is antibody P5A2 or an antigen-binding fragment thereof, and (b) detecting cells bound with the anti-idiotype antibody. In some of any such embodiments, the anti-idiotype antibody or antigen-binding fragment thereof is directly or indirectly labeled for detection.

In some embodiments, provided is a method of selecting cells from a cell population, including (a) contacting a cell population expressing a chimeric antigen receptor (CAR) containing a target antibody or a cell bound to a target antibody with the anti-idiotype antibody or antigen-binding fragment thereof according to any one of the embodiments described herein or conjugate according to any one of the embodiments described that specifically binds to a target antibody that is antibody C29 or an antigen-binding fragment thereof, wherein the target antibody is the antibody C29 or an antigen-binding fragment thereof, and (b) selecting cells bound with the anti-idiotype antibody. In some embodiments, the method includes (a) contacting a cell population expressing a chimeric antigen receptor (CAR) comprising a target antibody or a cell bound to a target antibody with the anti-idiotype antibody or antigen-binding fragment thereof of any one of the embodiments described or conjugate of any one of the embodiments described that specifically binds to a target antibody that is antibody P5A2 or an antigen-binding fragment thereof, wherein the target antibody is the antibody P5A2 or an antigen-binding fragment thereof; and (b) selecting cells bound with the anti-idiotype antibody.

In some instances, the cells bound with the anti-idiotype antibody are selected by affinity-based separation. In some aspects, the affinity-based separation is immunoaffinity-based separation. In some of any such embodiments, the affinity-based separation is by flow cytometry.

Also among the provided methods are methods for stimulating cells using the agents, such as stimulating cells containing a molecule such as a CAR that is or contains the target antibody recognized by the anti-idiotype antibody. In some aspects, the methods involve incubating an input composition containing cells expressing a chimeric antigen receptor (CAR) containing a target antibody that is the antibody C29 or an antigen-binding fragment thereof with the anti-idiotype antibody or antigen-binding fragment thereof according to any one of the embodiments described or the conjugate of any one of the embodiments described that specifically binds to a target antibody that is antibody C29 or an antigen-binding fragment thereof, thereby generating an output composition containing stimulated cells. In some embodiments, the method includes incubating an input composition containing cells expressing a chimeric antigen receptor (CAR) containing a target antibody that is the antibody P5A2 or an antigen-binding fragment thereof with the anti-idiotype antibody or antigen-binding fragment thereof of any one of the embodiments described or the conjugate of any one of the embodiments described that specifically binds to a target antibody that is antibody P5A2 or an antigen-binding fragment thereof, thereby generating an output composition containing stimulated cells.

In some embodiments, the methods result in proliferation, activation, stimulation, cytokine release, or other functional outcome such as upregulation of an activation marker or cytokine release or production, of cells expressing the chimeric receptor such as the CAR recognized by the anti-id antibody.

In some aspects, the CAR contains a target antibody that specifically binds to BCMA. In some embodiments, the target antibody is the antibody C29 or an antigen-binding fragment thereof. In some of any such embodiments, the anti-idiotype antibody or antigen-binding fragment thereof is the anti-idiotype antibody or antigen-binding fragment thereof according to any one of the embodiments described that specifically binds to a target antibody that is antibody C29 or an antigen-binding fragment thereof. In some cases, the target antibody is the antibody P5A2 or an antigen-binding fragment thereof. In some of any such embodiments, the anti-idiotype antibody or antigen-binding fragment thereof specifically binds to a target antibody that is antibody P5A2 of any one of embodiments described that specifically binds to a target antibody that is antibody P5A2 or an antigen-binding fragment thereof.

In some of any such embodiments, the target antibody or antigen-binding fragment contains a heavy chain variable region set forth in SEQ ID NO: 15 and/or a light chain variable region set forth in SEQ ID NO: 16. In some of any such embodiments, the target antibody or antigen-binding fragment contains a heavy chain variable region set forth in SEQ ID NO: 17 and/or a light chain variable region set forth in SEQ ID NO: 18.

In some embodiments, provided is a method of purifying an antibody or antigen-binding fragment thereof, including (a) contacting a composition or sample containing a target antibody that is the antibody C29 or an antigen-binding fragment thereof with the anti-idiotype antibody or antigen-binding fragment thereof according to any one of the embodiments described herein or conjugate according to any one of the embodiments described that specifically binds to a target antibody that is antibody C29 or an antigen-binding fragment thereof, and (b) isolating complexes containing the anti-idiotype antibody. In some embodiments, the method includes (a) contacting a composition or sample containing a target antibody that is the antibody P5A2 or an antigen-binding fragment thereof with the anti-idiotype antibody or antigen-binding fragment thereof of any one of the embodiments described or the conjugate of any one of the embodiments described that specifically binds to a target antibody that is antibody P5A2 or an antigen-binding fragment thereof, and (b) isolating complexes comprising the anti-idiotype antibody.

In some of any such embodiments, the antigen-binding fragment contains the variable heavy chain region and/or variable light chain region of the target antibody. In some embodiments, the antigen-binding fragment is a single chain fragment. In some aspects, the antigen-binding fragment is an scFv. In some of any such embodiments, the antigen-binding fragment is within or included in the antigen-binding domain of the extracellular portion of a chimeric antigen receptor (CAR).

In some of any such embodiments, the target antibody binds to BCMA. In some embodiments, the antigen-binding fragment of the target antibody is derived from antibody C29, optionally wherein the antigen-binding fragment of the target antibody contains a heavy chain variable region set forth in SEQ ID NO: 15 and/or a light chain variable region set forth in SEQ ID NO: 16. In some of any such embodiments, the antigen-binding fragment of the target antibody is a single chain variable fragment (scFv) derived from antibody C29, optionally wherein the scFv contains the sequence of amino acids set forth in SEQ ID NO:1 In some embodiments, the antigen-binding fragment of the target antibody is derived from antibody P5A2, optionally wherein the antigen-binding fragment of the target antibody contains a heavy chain variable region set forth in SEQ ID NO: 17 and/or a light chain variable region set forth in SEQ ID NO: 18. In some embodiments, the antigen-binding fragment of the target antibody is a single chain variable fragment (scFv) derived from antibody P5A2, optionally wherein the scFv contains the sequence of amino acids set forth in SEQ ID NO: 19.

In some embodiments, there is provided a method of depleting cells, comprising administering, to a subject, a composition comprising the anti-idiotype antibody or antigen-binding fragment thereof of according to any one of the embodiments described herein or conjugate according to any one of the embodiments described that specifically binds to a target antibody that is antibody C29 or an antigen-binding fragment thereof, wherein the subject has been administered a cell expressing a chimeric antigen receptor (CAR) comprising a target antibody that is the antibody C29 or an antigen-binding fragment thereof. In some embodiments, the method includes administering, to a subject, a composition comprising the anti-idiotype antibody or antigen-binding fragment thereof of any one of the embodiments described herein or conjugate of any one of the embodiments described that specifically binds to a target antibody that is antibody P5A2 or an antigen-binding fragment thereof, wherein the subject has been administered a cell expressing a chimeric antigen receptor (CAR) containing a target antibody that is the antibody P5A2 or an antigen-binding fragment thereof. In some embodiments, the depletion occurs via antibody-dependent cell-mediated cytotoxicity (ADCC).

In various embodiments, an antibody provided herein further comprises a detectable label, and a detectable label can be selected from the group consisting of a fluorescent label, a photochromic compound, a proteinaceous fluorescent label, a magnetic label, a radiolabel, and a hapten. When a fluorescent label is desired, the fluorescent label can be selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreen1, TagYFP, EYFP, Topaz, Venus, mCitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoeryhring (BPE), mCherry, HcRed1, Katusha, P3, Peridinin Chlorophyll (PerCP), mKate (TagFP635), TurboFP635, mPlum, and mRaspberry. In specific embodiments, a fluorescent label can be R-Phycoerythrin (PE) or Allophycocyanin (APC).

Also provided herein is a composition comprising an antibody provided herein and optionally a pharmaceutically acceptable carrier or vehicle.

Provided herein are polynucleotides encoding the heavy chain of an isolated antibody of an antibody provided herein. Further, polynucleotides encoding the light chain of an isolated antibody of an antibody provided herein are also provided. Vectors comprising a polynucleotide encoding the heavy chain of an isolated antibody of an antibody provided herein, and encoding the light chain of an isolated antibody of an antibody provided herein are also provided. Cells comprising such vectors are also provided, and in various embodiments, a cell comprises a cell selected from the group consisting of a CHO cell, a Sp2/0 cell, a rabbit cell and an E. coli cell.

Methods of making an isolated antibody provided herein are also provided and can comprise incubating a cell provided herein under suitable conditions.

A method of determining a number of cells expressing a C29 or P5A2 derived scFv is provided and can comprise contacting a sample with an isolated antibody that specifically binds the C29 or P5A2 derived scFv conjugated to a detectable label and determining the number of cells expressing the C29 or P5A2 derived scFv in the sample. In some embodiments, the isolated antibody that specifically binds the C29 or P5A2 derived scFv is an antibody provided herein or a humanized form thereof.

Also provided is a method of determining a number of cells presenting a polypeptide comprising an anti-BCMA scFv derived from C29 or P5A2, wherein the method comprises: (a) providing a sample comprising cells known or suspected to be presenting a polypeptide comprising an anti-BCMA scFv derived from C29 or P5A2; (b) contacting the sample with the isolated antigen binding molecule provided herein or a humanized form thereof under conditions that permit binding of the polypeptide and the antigen binding molecule; and (c) determining the number of cells presenting the polypeptide in the sample.

Provided herein is a method of determining the presence or absence of a polypeptide comprising an anti-BCMA scFv derived from C29 or P5A2, wherein the method comprises: (a) providing a sample known or suspected to comprise a polypeptide comprising an anti-BCMA scFv derived from C29 or P5A2; (b) contacting the sample with an isolated antigen binding molecule provided herein or a humanized form thereof under conditions that permit binding of the polypeptide and the antigen binding molecule; and (c) detecting the presence or absence of a polypeptide:antigen binding molecule complex. In an embodiment of the method, the sample is a formalin-fixed sample. In another embodiment, the C29 or P5A2 derived scFv is a component of a chimeric antigen receptor (CAR), and in further embodiments the cell expressing a C29 or P5A2 derived scFv CAR is an immune cell selected from the group consisting of CD8+ T cells, CD4+ T cells, tumor infiltrating lymphocytes (TILs), NK cells, TCR-expressing cells, dendritic cells, and NK-T cells. In some embodiments, the isolated antigen binding molecule is detectably labeled, and the detectable label can be selected from the group consisting of a fluorescent label, a photochromic compound, a proteinaceous fluorescent label, a magnetic label, a radiolabel, and a hapten. When the detectable label is a fluorescent label, the fluorescent label can be selected from the group consisting of an Atto dye, an Alexafluor dye, quantum dots, Hydroxycoumarin, Aminocouramin, Methoxycourmarin, Cascade Blue, Pacific Blue, Pacific Orange, Lucifer Yellow, NBD, R-Phycoerythrin (PE), PE-Cy5 conjugates, PE-Cy7 conjugates, Red 613, PerCP, TruRed, FluorX, Fluorescein, BODIPY-FL, Cy2, Cy3, Cy3B, Cy3.5, Cy5, Cy5.5, Cy7, TRITC, X-Rhodamine, Lissamine Rhocamine B, Texas Red, Allophycocyanin (APC), APC-Cy7 conjugates, Indo-1, Fluo-3, Fluo-4, DCFH, DHR, SNARF, GFP (Y66H mutation), GFP (Y66F mutation), EBFP, EBFP2, Azurite, GFPuv, T-Sapphire, Cerulean, mCFP, mTurquoise2, ECFP, CyPet, GFP (Y66W mutation), mKeima-Red, TagCFP, AmCyan1, mTFP1, GFP (S65A mutation), Midorishi Cyan, Wild Type GFP, GFP (S65C mutation), TurboGFP, TagGFP, GFP (S65L mutation), Emerald, GFP (S65T mutation), EGFP, Azami Green, ZsGreen1, TagYFP, EYFP, Topaz, Venus, mCitrine, YPet, TurboYFP, ZsYellow1, Kusabira Orange, mOrange, Allophycocyanin (APC), mKO, TurboRFP, tdTomato, TagRFP, DsRed monomer, DsRed2 (“RFP”), mStrawberry, TurboFP602, AsRed2, mRFP1, J-Red, R-phycoerythrin (RPE), B-phycoeryhring (BPE), mCherry, HcRed1, Katusha, P3, Peridinin Chlorophyll (PerCP), mKate (TagFP635), TurboFP635, mPlum, and mRaspberry. In a specific embodiment, the fluorescent label is R-Phycoerythrin (PE) or Allophycocyanin (APC). In some embodiments, the cell expressing a C29 or P5A2 derived scFv CAR is an immune cell, in which the immune cell is a T cell, and which can be disposed in vitro or in vivo. In some embodiments, the T cell is disposed in blood, extracted tissue, tissue grown ex vivo or cell culture media. In one embodiment, the T cell is an autologous T cell. In another embodiment, the T cell is an allogenic T cell.

In further aspects, provided herein are methods of making and using the anti-idiotypic antibodies. In a related aspect, provided herein are methods of quantifying or determining a number of cells expressing an anti-BCMA antibody comprising amino acid sequences of SEQ ID NOs: 15 and 16 or SEQ ID NOs: 17 and 18, comprising contacting the cells with the anti-idiotypic antibodies disclosed herein and determining the number of cells expressing the anti-BCMA antibody in the cells. In another aspect, the disclosure provides methods of determining a presence or absence of cells expressing an anti-BCMA antibody comprising amino acid sequences of SEQ ID NOs: 15 and 16 or SEQ ID NOs: 17 and 18, comprising contacting the cells with the isolated antibody and determining the presence or absence of cells expressing the anti-BCMA antibody in the cells. In yet another aspect, the instant disclosure provides methods of purifying an anti-BCMA antibody, or antigen-binding fragment thereof, wherein the anti-BMCA antibody comprises amino acid sequences of SEQ ID NOs: 15 and 16 or SEQ ID NOs: 17 and 18, the method comprising (a) contacting a sample comprising the anti-BCMA antibody with the anti-idiotypic antibodies disclosed herein to form a complex; and (b) separating or purifying the complex comprising the anti-BCMA antibody and the isolated antibody from the sample. In some embodiments, the anti-BCMA antibody is an scFv comprising an amino acid sequence of SEQ ID NO: 1 or SEQ ID NO: 19. In some embodiments, the cells express an anti-BCMA chimeric antigen receptor (CAR) comprising an amino acid sequence of SEQ ID NO: 1 or 19. In some embodiments, the cells are CAR T cells.

Further provided are methods of selecting cells that express an anti-BCMA CAR comprising an amino acid sequence of SEQ ID NO: 1 or 19, the method comprising contacting the cells with the anti-idiotypic antibodies described herein and selecting the cells bound with the anti-idiotypic antibody. In some embodiments, the anti-idiotypic antibodies further comprises a detectable label. In some embodiments, the cells bound with the anti-idiotypic antibodies are selected by affinity-based separation.

Also provided are methods for stimulating anti-BCMA CAR T cells comprising contacting the anti-BCMA CAR T cells with the antibodies described herein. In some embodiments, the antibodies are conjugated to beads or a solid surface. In certain embodiments, the anti-BCMA CAR T cells are present in a drug substance or drug product.

### BRIEF DESCRIPTION OF THE DRAWINGS

FIG. 1 shows sequence alignment of the VH and VL sequences of anti-BCMA antibody clone C29 and P5A2. The VH sequences of clones C29 and P5A2 share 95% identity and the VL sequences share a 99% identity. The CDR designations according to Kabat are underlined.

FIGS. 2A-2B show results of flow cytometry analysis showing binding specificity of the anti-BCMA anti-id antibody H6 for the anti-BCMA CAR clone P5A2, and not binding to anti-Flt3 CAR, anti-CD19 CAR or anti-CD70 CAR that were used as scFv or framework controls. NT: non-transduced.

### DETAILED DESCRIPTION

Provided herein are agents such as anti-idiotype (anti-id) antibodies and antigen-binding fragments (such as Fv, Fab, or single chain fragments, including scFvs) that specifically recognize anti-BCMA antibody moieties (such as anti-BCMA antibody moieties present in recombinant receptors, including chimeric antigen receptors). Also provided are uses and methods of use thereof, and compositions and articles of manufacture including such agents, including for specifically identifying, quantifying, selecting, isolating, purifying and/or stimulating and/or activating cells expressing or including the target antibodies or fragments such as anti-BCMA CAR T cells. In some embodiments, the provided antibodies can be used for specific identification, quantification and/or selection of various anti-BCMA CARs, such as CARs bound to or expressed on a cell surface, and can also be used to specifically activate cells expressing target CARs, such as CAR T cells. In some embodiments, provided are antibodies that are specific to the anti-BCMA antibody designated C29 or P5A2, or an antibody fragment derived therefrom, including antibodies and CARs containing variable regions derived from such antibodies, and/or an antibody containing an idiotope contained therein.

In some aspects, the provided anti-idiotype antibodies offer advantages compared to conventional reagents for detecting, identifying, manipulating and/or affecting and/or engineering cells that express a CAR, and in particular a CAR containing an anti-BCMA antibody scFv extracellular domain or one containing the recognized idiotype. In certain available methods, detection of the presence or absence or amount of CAR or CAR-expressing cells (and/or stimulation or manipulation of the CAR), in a sample, is carried out by assessing the presence or absence or amount of a surrogate molecule, such as one included in the construct encoding the CAR and thus serving as an indirect or surrogate marker for its expression.

The provided anti-idiotype antibodies and antigen-binding fragments in some embodiments overcome challenges of low binding affinity associated with target antibody ligands and non-specific binding associated with antibody reagents directed to target antibody constant regions, providing a reagent with both high affinity and specificity for its target antibody or antigen binding fragment thereof. In some embodiments, the provided antibodies exhibit greater specificity and binding affinity for their target antibodies or antigen-binding fragments, such as the anti-BCMA antibody designated C29 or P5A2, compared to BCMA-Fc and other reagents currently available for detecting or identifying the CAR comprising the anti-BCMA antibody.

In certain embodiments, the anti-idiotype antibodies are multispecific. Among the multispecific binding molecules are multispecific antibodies, including, e.g. bispecific. Multispecific binding partners, e.g., antibodies, have binding specificities for at least two different sites, which may be in the same or different antigens. In certain embodiments, one of the binding specificities is for an anti-BCMA antibody moiety and the other is for another antigen. In certain embodiments, bispecific antibodies may bind to two different epitopes of an anti-BCMA antibody moiety. Bispecific antibodies may also be used to localize cytotoxic agents to cells which express an anti-BCMA antibody moiety on their surface, such as anti-BCMA CAR T cells. Bispecific antibodies can be prepared as full length antibodies or antibody fragments. Among the bispecific antibodies are multispecific single-chain antibodies, e.g., diabodies, triabodies, and tetrabodies, tandem di-scFvs, and tandem tri-scFvs.

Furthermore, in certain embodiments, anti-idiotype antibodies and antigen-binding fragments may be selected as agonists or antagonists of chimeric receptors comprising their target antibodies or antigen-binding fragments, allowing for selective detection, isolation, ablation and/or depletion (for example, killing via antibody-dependent cell-mediated cytotoxicity, ADCC), and/or stimulation or activation of cells with such chimeric receptors bound to or expressed on their surface. In some embodiments, the anti-idiotype antibodies can be humanized or fully human antibodies. Provided herein are anti-idiotype antibody agonists that exhibit activity to stimulate, such as activate, a CAR containing an extracellular binding domain derived from anti-BCMA antibody designated C29 or P5A2. In some aspects, such antibodies can be used in methods of stimulating and expanding specific CAR-expressing cells, including in processes for generating and preparing the CAR-expressing cells. In some embodiments, the methods of stimulating and expanding can be in vitro methods. In some embodiments, the methods of stimulating and expanding can be incorporated into the CAR T manufacturing process. In some embodiments, the methods of stimulating and expanding can be in vivo methods. In some embodiments, the anti-idiotype antibodies can be humanized or fully human antibodies.

Also provided herein are nucleic acids encoding the provided anti-idiotype antibodies and fragments, and cells, such as recombinant cells, expressing and for production of these anti-idiotype antibodies and fragments. Also provided are methods of making and using the anti-idiotype antibodies and fragments, as well as cells expressing or containing the anti-idiotype antibodies and fragments.

The scFv portion of some chimeric antigen receptors (CARs) is derived from the fully-human antibody clones C29 or P5A2 with high affinity to BCMA. The present disclosure provides reagents to detect anti-BCMA CARs comprising an scFv portion derived from antibody clones C29 or P5A2. Disclosed herein are anti-idiotype antibodies that specifically bind to anti-BCMA clones C29 or P5A2, and anti-BCMA molecules derived from C29 or P5A2. Anti-id antibodies from C29 derived molecules disclosed herein demonstrate specific high affinity binding to chimeric antigen receptors (CARs) comprising a C29 derived scFv. One unlimiting example of the C29 derived scFv comprises the amino acid sequence of SEQ ID NO: 1, which was also found to cross-react with, and recognize, antibody clone P5A2. One unlimiting example of the P5A2 derived scFv comprises the amino acid sequence of SEQ ID NO: 19.

When conjugated to a bright fluorochrome, the anti-id antibodies disclosed herein stain cells expressing chimeric antigen receptors (CARs) comprising a C29 or P5A2 derived scFv with a high MFI and low background. The antibodies described herein can be used in a method to detect anti-BCMA CAR expression. These antibodies can be used for identification by both flow cytometry and immunohistochemistry. These antibodies can also be used in the context of, for example, non-clinical research studies, in the manufacturing of immune cells comprising a C29 or P5A2 derived scFv, as clinical flow-based pharmacokinetic reagents, and in clinical immunogenicity studies.

C29 is a BCMA monoclonal antibody that recognizes human BCMA. Single chain variable fragments (scFv) formed from C29 comprise the targeting component of some chimeric antigen receptors (CARs). In some embodiments, the scFv derived from the BCMA monoclonal antibody C29 comprises a part of the BCMA monoclonal antibody C29 immunoglobulin gamma 1 heavy chain (SEQ ID NO: 15) and a part of anti-BCMA monoclonal antibody C29 immunoglobulin kappa light chain (SEQ ID NO: 16), linked together by a flexible linker. In some embodiments, the scFv comprises the variable fragments of the anti-BCMA monoclonal antibody C29 immunoglobulin gamma 1 heavy chain and the variable fragments of the anti-BCMA monoclonal antibody C29 immunoglobulin kappa light chain linked together by a flexible linker.

P5A2 is also a BCMA monoclonal antibody that recognizes BCMA. Single chain variable fragments formed from P5A2 comprise the targeting component of some chimeric antigen receptors. In some embodiments, the scFv derived from the BCMA monoclonal antibody P5A2 comprises a part of the BCMA monoclonal antibody P5A2 immunoglobulin gamma 1 heavy chain (SEQ ID NO: 17) and a part of anti-BCMA monoclonal antibody P5A2 immunoglobulin kappa light chain (SEQ ID NO: 18), linked together by a flexible linker. In some embodiments, the scFv comprises the variable fragments of the anti-BCMA monoclonal antibody P5A2 immunoglobulin gamma 1 heavy chain and the variable fragments of the anti-BCMA monoclonal antibody P5A2 immunoglobulin kappa light chain linked together by a flexible linker.

The anti-BCMA monoclonal antibody C29 immunoglobulin gamma 1 heavy chain variable domain comprises the amino acid sequence:

(SEQ ID NO: 15)
EVQLLESGGGLVQPGGSLRLSCAASGFTFSSYPMSWVRQA
PGKGLEWVSAIGGSGGSLPYADSVKGRFTISRDNSKNTLY
LQMNSLRAEDTAVYYCARYWPMDIWGQGTLVTVSS

The anti-BCMA monoclonal antibody C29 immunoglobulin kappa light chain variable domain comprises the amino acid sequence:

(SEQ ID NO: 16)
EIVLTQSPGTLSLSPGERATLSCRASQSVSSSYLAWYQQK
PGQAPRLLMYDASIRATGIPDRFSGSGSGTDFTLTISRLE
PEDFAVYYCQQYQSWPLTFGQGTKVEIK

The anti-BCMA monoclonal antibody P5A2 immunoglobulin gamma 1 heavy chain variable domain comprises the amino acid sequence:

(SEQ ID NO: 17)
EVQLLESGGGLVQPGGSLRLSCAASGFTFSSYAMNWVRQA
PGKGLEWVSAISDSGGSTYYADSVKGRFTISRDNSKNTLY
LQMNSLRAEDTAVYYCARYWPMDIWGQGTLVTVSS

The anti-BCMA monoclonal antibody P5A2 immunoglobulin kappa light chain variable domain comprises the amino acid sequence:

(SEQ ID NO: 18)
EIVLTQSPGTLSLSPGERATLSCRASQSVSSSYLAWYQQK
PGQAPRLLMYDASIRATGIPDRFSGSGSGTDFTLTISRLE
PEDFAVYYCQQYGSWPLTFGQGTKVEIK

An exemplary C29 derived scFv comprises the amino acid sequence:

(SEQ ID NO: 1)
EVQLLESGGGLVQPGGSLRLSCAASGFTFSSYPMSWVRQA
PGKGLEWVSAIGGSGGSLPYADSVKGRFTISRDNSKNTLY
LQMNSLRAEDTAVYYCARYWPMDIWGQGTLVTVSSGGGGS
GGGGSGGGGSEIVLTQSPGTLSLSPGERATLSCRASQSVS
SSYLAWYQQKPGQAPRLLMYDASIRATGIPDRFSGSGSGT
DFTLTISRLEPEDFAVYYCQQYQSWPLTFGQGTKVEIK

An exemplary P5A2 derived scFv comprises the amino acid sequence:

(SEQ ID NO: 19)
EVQLLESGGGVQPGGSLRLSCAASGFTFSSYAMNWVRQAP
GKGLEWVSAISDSGGSTYYADSVKGRFTISRDNSKNTLYL
QMNSLRAEDTAVYYCARYWPMDIWGQGTLVTVSSGGGGSG
GGGSGGGGSEIVLTQSPGTLSLSPGERATLSCRASQSVSS
SYLAWYQQKPGQAPRLLMYDASIRATGIPDRFSGSGSGTD
FTLTISRLEPEDFAVYYCQQYGSWPLTFGQGTKVEIK

In some embodiments, the scFv comprises at least a part of amino acid sequences of SEQ ID NO: 15 and/or SEQ ID NO: 16. In some embodiments, the scFv comprises at least a part of amino acid sequences of SEQ ID NO: 15 and/or SEQ ID NO: 16, with or without the signal sequence. In some embodiments, the scFv comprises at least 50%, at least 60%, at least 70%, at least 80%, at least 90%, at least 95%, at least 97%, at least 98% or at least 99% sequence identity with the amino acid sequence of SEQ ID NO: 15 and/or SEQ ID NO: 16. In some embodiments, the scFv comprises at least a part of amino acid sequences of SEQ ID NO: 17 and/or SEQ ID NO: 18. In some embodiments, the scFv comprises at least a part of amino acid sequences of SEQ ID NO: 17 and/or SEQ ID NO: 18, with or without the signal sequence. In some embodiments, the scFv comprises at least 50%, at least 60%, at least 70%, at least 80%, at least 90%, at least 95%, at least 97%, at least 98% or at least 99% sequence identity with the variable region of amino acid sequence of SEQ ID NO: 17 and/or SEQ ID NO: 18. In some embodiments, the scFv comprises at least 50%, at least 60%, at least 70%, at least 80%, at least 90%, at least 95%, at least 97%, at least 98%, at least 99%, or 100% sequence identity with the amino acid sequence of SEQ ID NO: 1. In some embodiments, the scFv comprises at least 50%, at least 60%, at least 70%, at least 80%, at least 90%, at least 95%, at least 97%, at least 98%, at least 99%, or 100% sequence identity with the amino acid sequence of SEQ ID NO: 19. Disclosed herein are antigen binding molecules, including antibodies, that specifically bind to the anti-BCMA scFv derived from C29 or P5A2, as well as molecules comprising these sequences and cells presenting such molecules. Humanized forms of the antigen binding molecules also form as aspect of the disclosure. Applications and uses of these antigen binding molecules are also disclosed.

The section headings used herein are for organizational purposes only and are not to be construed as limiting the subject matter described.

### I. Antigen Binding Molecules and Polynucleotides Encoding Same

An “antigen binding domain” as used herein means any polypeptide that binds a specified target antigen, for example the specified target antigen can be the BCMA protein or fragment thereof (referred to interchangeably herein as a “BCMA antigen”, “BCMA target antigen”, or “BCMA target”). In the context of an anti-idiotype antibody of the present disclosure, the target antigen is an antigen binding molecule that specifically binds BCMA (e.g., antibody clones C29 or P5A2 and antigen binding molecules derived from or related to C29 or P5A2, including scFvs).

In some embodiments, the antigen binding domain binds to a BCMA antigen on a tumor cell. In some embodiments, the antigen binding domain binds to a BCMA antigen on a cell involved in a hyperproliferative disease or to a viral or bacterial antigen.

Antigen binding domains include, but are not limited to, antibody binding regions that are immunologically functional fragments. The term “immunologically functional fragment” (or “fragment”) of an antigen binding domain is a species of antigen binding domain comprising a portion (regardless of how that portion is obtained or synthesized) of an antibody that lacks at least some of the amino acids present in a full-length chain, but which is still capable of specifically binding to a target antigen. Such fragments are biologically active in that they bind to the target antigen and can compete with other antigen binding domains, including intact antibodies, for binding to a given epitope. In some embodiments, the fragments are neutralizing fragments. In some embodiments, the fragments can block or reduce the activity of an anti-BCMA CAR (e.g., a blocking effect). In some embodiments, the fragments can antagonize the activity of an anti-BCMA CAR.

In specific embodiments, an anti-id antibody of the instant disclosure is an antibody identified herein as Clone H6 and comprises the heavy and light chain amino acids, variable regions, CDR sequences and nucleotide sequences encoding such sequences, as provided and labeled herein.

Immunologically functional immunoglobulin fragments include, but are not limited to, scFv fragments, Fab fragments (Fab′, F(ab′)2, and the like), one or more complementarity determining regions (“CDRs”), a diabody (heavy chain variable domain on the same polypeptide as a light chain variable domain, connected via a short peptide linker that is too short to permit pairing between the two domains on the same chain), domain antibodies, bivalent antigen binding domains (comprises two antigen binding sites), multispecific antigen binding domains, and single-chain antibodies. These fragments can be derived from any mammalian source, including but not limited to human, mouse, rat, camelid or rabbit. As will be appreciated by one of skill in the art, an antigen binding domain can include non-protein components.

The variable regions typically exhibit the same general structure of relatively conserved framework regions (FR) joined by the 3 hypervariable regions (CDRs). The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3.

In some embodiments, antigen binding domains comprise one or more complementarity binding regions (CDRs) present in the full-length light or heavy chain of an antibody, and in some embodiments comprise a single heavy chain and/or light chain or portion thereof. These fragments can be produced by recombinant DNA techniques or can be produced by enzymatic or chemical cleavage of antigen binding domains, including intact antibodies.

In some embodiments, the antigen binding domain is an antibody of fragment thereof, including one or more of the complementarity determining regions (CDRs) thereof. In some embodiments, the antigen binding domain is a single chain variable fragment (scFv), comprising light chain CDRs CDR1, CDR2 and CDR3, and heavy chain CDRs CDR1, CDR2 and CDR3.

The assignment of amino acids to each of the framework, CDR, and variable domains is typically in accordance with numbering schemes of Kabat numbering (see, e.g., Kabat et al. in Sequences of Proteins of Immunological Interest, 5th Ed., NIH Publication 91-3242, Bethesda Md. 1991), Chothia numbering (see, e.g., Chothia & Lesk, (1987), J Mol Biol 196: 901-917; Al-Lazikani et al., (1997) J Mol Biol 273: 927-948; Chothia et al., (1992) J Mol Biol 227: 799-817; Tramontano et al., (1990) J Mol Biol 215(1): 175-82; and U.S. Pat. No. 7,709,226), contact numbering, or the AbM scheme (Antibody Modeling program, Oxford Molecular).

Accordingly, in some embodiments, the CDRs of the anti-idiotype antibodies presented herein are numbered according to the Kabat numbering scheme. In other embodiments, the CDRs of the anti-idiotype antibodies presented herein are numbered according to the Chothia numbering scheme. In other embodiments, the CDRs of the anti-idiotype antibodies presented herein are numbered according to the contact numbering scheme. In other embodiments, the CDRs of the anti-idiotype antibodies presented herein are numbered according to the AbM numbering scheme.

Humanized antibodies are described herein and can be prepared by known techniques. In some embodiments, a humanized monoclonal antibody comprises the variable domain of an anti-id antibody (or all or part of the antigen binding site thereof) and a constant domain derived from a human antibody. Alternatively, a humanized antibody fragment can comprise an antigen binding site of a murine or rabbit monoclonal antibody and a variable domain fragment (lacking the antigen binding site) derived from a human antibody. Procedures for the production of engineered monoclonal antibodies include those described in, e.g., Riechmann et al., (1988) Nature 332:323, Liu et al., (1987) Proc. Nat. Acad. Sci. USA 84:3439, Larrick et al., (1989) Bio/Technology 7:934, and Winter et al., (1993) TIPS 14:139. In some embodiments, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557; Padlan et al., (1995) FASEB J. 9:133-39; Tamura et al., (2000) J. Immunol. 164:1432-41; Zhang et al., (2005) Mol. Immunol. 42(12):1445-1451; Hwang et al., Methods. (2005) 36(1):35-42; Dall'Acqua et al., (2005) Methods 36(1):43-60; and Clark, (2000) Immunology Today 21(8):397-402.

Variants of the anti-idiotype antibodies are also within the scope of the disclosure, e.g., variable light and/or variable heavy chains that each have at least 70-80%, 80-85%, 85-90%, 90-95%, 95-97%, 97-99%, or above 99% identity to the amino acid sequences of the antigen binding domain sequences described herein. In some embodiments, the anti-idiotype antibody is at least about 75%, at least about 85%, at least about 85%, at least about 90%, at least about 95%, at least about 96%, at least about 97%, at least about 98%, at least about 99%, or 100% identical to a heavy chain variable region sequence provided in Table 1a and/or a light chain variable sequence provided in Table 1b.

In some instances, such molecules include at least one heavy chain and one light chain, whereas in other instances the variant forms contain two variable light chains and two variable heavy chains (or subparts thereof). A skilled artisan will be able to determine suitable variants of the anti-idiotype antibodies as set forth herein using well-known techniques. In certain embodiments, one skilled in the art can identify suitable areas of the molecule that can be changed without destroying activity by targeting regions not believed to be important for activity.

An anti-id antibody of the present disclosure can also be a fully human monoclonal antibody. Fully human monoclonal antibodies can be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

An anti-id antibody that specifically binds to anti-BCMA clones C29 or P5A2 and anti-BCMA molecules derived from C29 or P5A2 is said to be “selective” when it binds to one target more tightly than it binds to a second target.

An anti-id antibody that specifically binds to anti-BCMA clones C29 or P5A2 and anti-BCMA molecules derived from C29 or P5A2 is said to “specifically bind” its target antigen (e.g., mouse C29 or P5A2 and molecules derived from C29 or P5A2) when the dissociation constant (Kd) is ˜1 nM. The antigen binding domain specifically binds antigen with “high affinity” when the Kd is 1-5 nM, and with “very high affinity” when the Kd is 0.1-0.5 nM. In one embodiment, the antigen binding domain has a Kd of ˜1 nM. In one embodiment, the off-rate is <1×10⁻⁵. In other embodiments, the antigen binding domains will bind to mouse C29 or P5A2 and molecules derived from C29 or P5A2 with a Kd of between about 1×10⁻⁷ M and 1×10⁻¹² M, and in yet another embodiment the antigen binding domains will bind with a Kd between about 1×10⁻⁵ and 1×10⁻¹².

As provided herein, the anti-id antibodies of the present disclosure specifically bind mouse C29 or P5A2 and molecules derived from C29 or P5A2 (e.g., murine C29 or P5A2, humanized C29 or P5A2, C29 or P5A2 derived CARs). In certain embodiments, the anti-id antibodies of the present disclosure bind mouse C29 or P5A2 and molecules derived from C29 or P5A2 with a KD of less than 1×10⁻⁶ M, less than 1×10⁻⁷ M, less than 1×10⁻⁸ M, or less than 1×10⁻⁹ M. In one particular embodiment, the anti-id antibodies bind mouse C29 or P5A2 and molecules derived from C29 or P5A2 with a KD of less than 1×10⁻⁷ M. In another embodiment, the anti-id antibodies bind mouse C29 or P5A2 and molecules derived from C29 or P5A2 with a KD of less than 1×10⁻⁸ M. In some embodiments, the anti-id antibodies bind mouse C29 or P5A2 and molecules derived from C29 or P5A2 with a Kd of about 1×10⁻⁷ M, about 2×10⁻⁷ M, about 3×10⁻⁷ M, about 4×10⁻⁷ M, about 5×10⁻⁷ M, about 6×10⁻⁷ M, about 7×10⁻⁷ M, about 8×10⁻⁷ M, about 9×10⁻⁷ M, about 1×10⁻⁸ M, about 2×10⁻⁸ M, about 3×10⁻⁸ M, about 4×10⁻⁸ M, about 5×10⁻⁸M, about 6×10⁻⁸M, about 7×10⁻⁸M, about 8×10⁻⁸ M, about 9×10⁻⁸ M, about 1×10⁻⁹ M, about 2×10⁻⁹ M, about 3×10⁻⁹ M, about 4×10⁻⁹ M, about 5×10⁻⁹ M, about 6×10⁻⁹ M, about 7×10⁻⁹ M, about 8×10⁻⁹ M, about 9×10⁻⁹ M, about 1×10⁻¹⁰ M, or about 5×10⁻¹⁰ M. In certain embodiments, the Kd is calculated as the quotient of K_{off}/Kₒₙ, and the Kₒₙ and K_{off} are determined using a monovalent antibody, such as a Fab fragment, as measured by, e.g., BIAcore® surface plasmon resonance technology. In other embodiments, the Kd is calculated as the quotient of K_{off}/Kₒₙ, and the Kₒₙ and K_{off} are determined using a bivalent antibody, such as a Fab fragment, as measured by, e.g., BIAcore® surface plasmon resonance technology.

In some embodiments, the anti-id antibodies bind mouse C29 or P5A2 and molecules derived from C29 or P5A2 with an association rate (kₒₙ) of less than 1×10⁻⁴ M⁻¹ s⁻¹, less than 2×10⁻⁴ M⁻¹ s⁻¹ less than 3×10⁻⁴ M⁻¹ s⁻¹ less than 4×10⁻⁴ M⁻¹ s⁻¹ less than 5×10⁻⁴ M⁻¹ s⁻¹ less than 7×10⁴ M⁻¹ s⁻¹ less than 8×10⁻⁴ M⁻¹ s⁻¹ less than 9×10⁴ M⁻¹ s⁻¹ less than 1×10⁵ M⁻¹ s⁻¹, less than 2×10⁻⁵ M⁻¹ s⁻¹ less than 3×10⁻⁵ M⁻¹ s⁻¹ less than 4×10⁻⁵ M⁻¹ s⁻¹ less than 5×10⁻⁵ M⁻¹ s⁻¹ less than 6×10⁻⁵ M⁻¹ s⁻¹ less than 7×10⁻⁵ M⁻¹ s⁻¹ less than 8×10⁻⁵ M⁻¹ s⁻¹ less than 9×10⁻⁵ M⁻¹ s⁻¹, less than 1×10⁻⁶ M⁻¹ s⁻¹, less than 2×10⁻⁶ M⁻¹ s⁻¹, less than 3×10⁻⁶ M⁻¹ s⁻¹, less than 4×10⁻⁶ M⁻¹ s⁻¹ less than 5×10⁻⁶ M⁻¹ s⁻¹ less than 6×10⁻⁶ M⁻¹ s⁻¹ less than 7×10⁻⁶ M⁻¹ s⁻¹, less than 8×10⁻⁶ M⁻¹ s⁻¹, less than 9×10⁻⁶ M⁻¹ s⁻¹, or less than 1×10⁻⁷ M⁻¹ s⁻¹. In certain embodiments, the kₒₙ is determined using a monovalent antibody, such as a Fab fragment, as measured by, e.g., BiAcore® surface plasmon resonance technology. In other embodiments, the kₒₙ is determined using a bivalent antibody as measured by, e.g., BIAcore® surface plasmon resonance technology.

In some embodiments, the anti-id antibodies bind mouse C29 or P5A2 and molecules derived from C29 or P5A2 with an dissociation rate (k_{off}) of less than 1×10⁻² s⁻¹, less than 2×10⁻² s⁻¹, less than 3×10⁻² s⁻¹, less than 4×10⁻² s⁻¹, less than 5×10⁻² s⁻¹, less than 6×10⁻² s⁻¹, less than 7×10⁻² s⁻¹, less than 8×10⁻² s⁻¹, less than 9×10⁻² s⁻¹, less than 1×10⁻³ s⁻¹, less than 2×10⁻³ s⁻¹, less than 3×10⁻³ s⁻¹, less than 4×10⁻³ s⁻¹, less than 5×10⁻³ s⁻¹, less than 6×10⁻³ s⁻¹, less than 7×10⁻³ s⁻¹, less than 8×10⁻³ s⁻¹, less than 9×10⁻³ s⁻¹, less than 1×10⁻⁴ s⁻¹, less than 2×10⁻⁴ s⁻¹, less than 3×10⁻⁴ s⁻¹, less than 4×10⁻⁴ s⁻¹, less than 5×10⁻⁴ s⁻¹, less than 6×10⁻⁴ s⁻¹ less than 7×10⁻⁴ s⁻¹, less than 8×10⁻⁴ s⁻¹, less than 9×10⁻⁴ s⁻¹, less than 1×10⁻⁵ s⁻¹, or less than 5×10⁻⁵ s⁻¹. In certain embodiments, the k_{off} is determined using a monovalent antibody, such as a Fab fragment, as measured by, e.g., BIAcore® surface plasmon resonance technology. In other embodiments, the k_{off} is determined using a bivalent antibody as measured by, e.g., BIAcore® surface plasmon resonance technology.

Provided herein are anti-idiotype antibodies that specifically bind to anti-BCMA clones C29 or P5A2 and antigen binding molecules derived from C29 or P5A2, comprising a variable heavy chain (VH), wherein the amino acid sequence or polynucleotide sequence of the VH is selected from the VH sequences presented in Table 1a.

TABLE la
Heavy Chain Variable Regions (VH)

| | | |
|---|---|---|
| Clone | VH Sequence | SEQ ID NO: |

Amino Acid Sequence

| | | |
|---|---|---|
| H6 | QVTLKESGPGILQPSQTLSL | 2 |
| | TCSFSGFSLNTSAMGVGWIR | |
| | QPSGKGLEWLAHIWWNDDHY | |
| | YNPALKSRVTISKDASNNQV | |
| | FLKIASVVTADTATYYCARS | |
| | IAPVVPTPFAYWGQGTLVTV | |
| | SA | |
| F3 | EVQLQESGPGILQPSQTLSL | 20 |
| | TCSFSGFSLSTSGMNVGWIR | |
| | QPSGKGLEWLAHMWNDDNYY | |
| | NPALKSRLTISKDPSNNQVF | |
| | LKIASVVTADTATYFCARLE | |
| | ESTTITTAFAYWGQGTLVTV | |
| | SA | |
| E10 | QVQLQQSGPGLVQPSQSLSI | 33 |
| | PCTVSGFSLTSYDVHWVRQS | |
| | PGKGLEWLGVIWSGGSTGYN | |
| | AIFRSRLSINKDNSKSQVFF | |
| | KMTSLQPDDTAIYYCANFSP | |
| | WYLPSYWDQGTLVTVSA | |

Polynucleotide Sequence

| | | |
|---|---|---|
| H6 | CAGGTTACTCTGAAAGAGTC | 3 |
| | TGGCCCTGGGATATTGCAGC | |
| | CCTCCCAGACCCTCAGTCTG | |
| | ACTTGTTCTTTTTCTGGGTT | |
| | TTCACTGAACACTTCTGCTA | |
| | TGGGTGTAGGCTGGATTCGT | |
| | CAGCCTTCAGGGAAGGGTCT | |
| | GGAGTGGCTGGCACACATTT | |
| | GGTGGAATGATGATCACTAC | |
| | TATAATCCAGCCCTGAAAAG | |
| | CCGGGTCACAATCTCCAAGG | |
| | ATGCCTCCAACAACCAGGTA | |
| | TTCCTCAAGATCGCCAGTGT | |
| | GGTCACTGCAGATACTGCCA | |
| | CATACTACTGTGCTCGATCT | |
| | ATTGCTCCGGTAGTGCCTAC | |
| | ACCGTTTGCTTACTGGGGCC | |
| | AAGGGACTCTGGTCACTGTC | |
| | TCTGCA | |
| F3 | GAGGTGCAGCTGCAGGAGTC | 21 |
| | TGGCCCTGGGATATTGCAGC | |
| | CCTCCCAGACCCTCAGTCTG | |
| | ACTTGTTCTTTCTCTGGGTT | |
| | TTCACTGAGCACTTCTGGTA | |
| | TGAATGTAGGCTGGATTCGT | |
| | CAGCCTTCAGGGAAGGGTCT | |
| | GGAGTGGCTGGCACACATGT | |
| | GGAATGATGATAACTACTAT | |
| | AACCCAGCCCTGAAAAGCCG | |
| | GCTCACAATCTCCAAGGATC | |
| | CCTCCAACAACCAGGTATTC | |
| | CTCAAGATCGCCAGTGTGGT | |
| | CACTGCAGATACTGCCACAT | |
| | ACTTCTGTGCTCGATTAGAG | |
| | GAATCTACTACGATTACGAC | |
| | GGCTTTTGCTTATTGGGGCC | |
| | AAGGGACTCTGGTCACTGTC | |
| | TCCGCAG | |
| E10 | CAGGTGCAGCTGCAGCAGTC | 34 |
| | AGGACCTGGCCTAGTGCAGC | |
| | CCTCACAGAGCCTGTCCATC | |
| | CCCTGCACAGTCTCTGGTTT | |
| | CTCATTAACTAGCTATGATG | |
| | TACATTGGGTTCGCCAGTCT | |
| | CCAGGAAAGGGTCTGGAGTG | |
| | GCTGGGAGTGATCTGGAGTG | |
| | GTGGCAGCACAGGCTATAAT | |
| | GCAATTTTCAGATCCAGACT | |
| | GAGCATCAACAAGGACAACT | |
| | CCAAGAGCCAAGTTTTCTTT | |
| | AAAATGACCAGTCTGCAACC | |
| | TGATGACACAGCCATATATT | |
| | ACTGTGCCAATTTCTCCCCG | |
| | TGGTACCTCCCTTCTTACTG | |
| | GGACCAAGGGACTCTGGTCA | |
| | CTGTCTCTGCAG | |

Provided herein are anti-idiotype antibodies that specifically bind to anti-BCMA clones C29 or P5A2, comprising a variable light chain (VL), wherein the amino acid sequence or polynucleotide sequence of the VL is selected from the VL sequences presented in Table 1b.

TABLE 1b
Light Chain Variable Regions

| | | |
|---|---|---|
| Clone | VL Sequence | SEQ ID NO: |

Amino Acid Sequence

| | | |
|---|---|---|
| H6 | DIQMTQSSSSFSVSLGDRVT | 4 |
| | ITCKASEDIYNRLAWFQQKP | |
| | GNAPRLLISGATRLETGVPS | |
| | RFSGSGSGKDYTLSITSLQT | |
| | EDVGTYYCQQYWSSSWTFGG | |
| | GTKLEIK | |
| F3 | EIVVTQSPALMAASPGEKVT | 22 |
| | ITCSVSSSISSSNLHWYQQK | |
| | SETSPK | |
| | PWIYGTSNLASGVPVRFSGS | |
| | GSGTSYSLTISSMEAEDAAT | |
| | YYCQQ | |
| | WSSYPLTFGAGTKLEIK | |
| E10 | QIVLTQSPAIMSASPGEKVT | 35 |
| | ITCSASSNVSYMHWFQQKPG | |
| | TSPKL | |
| | WIYSTSNLASGVPARFSGSG | |
| | SGTSYSLTISRMEAEDAATY | |
| | YCQQ | |
| | RSSYPLTFGAGTKLELK | |

Polynucleotide Sequence

| | | |
|---|---|---|
| H6 | GACATCCAGATGACACAATC | 5 |
| | TTCATCCTCCTTTTCTGTAT | |
| | CTCTAGGAGACAGAGTCACC | |
| | ATTACTTGCAAGGCAAGTGA | |
| | GGACATATATAATCGGTTAG | |
| | CCTGGTTTCAGCAGAAACCA | |
| | GGAAATGCTCCTAGGCTCTT | |
| | AATATCTGGTGCAACCAGAT | |
| | TGGAAACTGGGGTTCCTTCA | |
| | AGATTCAGTGGCAGTGGATC | |
| | TGGAAAGGATTACACTCTCA | |
| | GCATTACCAGTCTTCAGACT | |
| | GAAGATGTTGGTACTTATTA | |
| | CTGTCAACAGTATTGGAGTT | |
| | CTTCGTGGACGTTCGGTGGA | |
| | GGCACCAAGCTGGAAATCAA | |
| | A | |
| F3 | GAAATTGTGGTCACCCAGTC | 23 |
| | TCCAGCACTCATGGCTGCAT | |
| | CTCCGGGGGAGAAGGTCACC | |
| | ATCACCTGCAGTGTCAGCTC | |
| | TAGTATAAGTTCCAGCAATT | |
| | TGCACTGGTACCAGCAGAAG | |
| | TCAGAAACCTCCCCCAAACC | |
| | CTGGATTTATGGCACATCCA | |
| | ACCTGGCTTCTGGAGTCCCT | |
| | GTTCGCTTCAGTGGCAGTGG | |
| | ATCTGGGACCTCTTATTCTC | |
| | TCACAATCAGCAGCATGGAG | |
| | GCTGAAGATGCTGCCACTTA | |
| | TTATTGTCAACAGTGGAGTA | |
| | GTTACCCGCTCACGTTCGGT | |
| | GCTGGGACCAAGCTGGAAAT | |
| | AAAAC | |
| E10 | CAAATTGTTCTCACCCAGTC | 36 |
| | TCCAGCAATCATGTCTGCTT | |
| | CTCCAGGGGAGAAGGTCACC | |
| | ATAACCTGCAGTGCCAGCTC | |
| | AAATGTCAGTTACATGCACT | |
| | GGTTCCAGCAGAAGCCAGGC | |
| | ACTTCTCCCAAACTCTGGAT | |
| | TTATAGCACATCCAACCTGG | |
| | CTTCTGGAGTCCCTGCTCGC | |
| | TTCAGTGGCAGTGGATCTGG | |
| | GACCTCTTACTCTCTCACAA | |
| | TCAGCCGAATGGAGGCTGAA | |
| | GATGCTGCCACTTATTACTG | |
| | CCAGCAAAGGAGTAGTTACC | |
| | CACTCACGTTCGGTGCTGGG | |
| | ACTAAGCTGGAGCTGAAAC | |

Provided herein are anti-idiotype antibodies that specifically bind to anti-BCMA clones C29 or P5A2 and antigen binding molecules derived from C29 or P5A2, wherein anti-id antibodies comprise a variable heavy chain (VH) and a variable light chain (VL), wherein the amino acid sequence or polynucleotide sequence of the VH is selected from the VH sequences presented in Table 1a; and wherein the amino acid sequence or polynucleotide sequence of the VL is selected from the VL sequences presented in Table 1b.

In some embodiments, the anti-idiotype antibodies that specifically bind to anti-BCMA clones C29 or P5A2 and antigen binding molecules derived from C29 or P5A2, comprise a VH CDR 1, CDR2, and CDR3 of a VH sequence presented in Table 1a. In some embodiments, the VH CDR 1, CDR2, and CDR3 are selected from a CDR sequence presented in Table 1c.

TABLE 1c
Heavy Chain CDRs

| Clone | CDR H1 | CDR H2 | CDR H3 |
|---|---|---|---|
| H6 | TSAMGVG | HIWWND | SIAPVV |
| | (SEQ ID | DHYYNP | PTPFAY |
| | NO: 6) | ALKS | (SEQ ID |
| | (Kabat) | (SEQ ID | NO: 11) |
| | GFSLNTSAM | NO: 9) | (Kabat/ |
| | (SEQ ID | (Kabat) | Chothia) |
| | NO: 7) | WWNDD | |
| | (Chothia) | (SEQ ID | |
| | GFSLNTS | NO: 10) | |
| | AMGVG | (Chothia) | |
| | (SEQ ID | | |
| | NO: 8) | | |
| | (Extended) | | |
| F3 | TSGMNVG | HMWNDDN | LEESTTI |
| | (SEQ ID | YYNPALKS | TTAFAY |
| | NO: 24) | (SEQ ID | (SEQ ID |
| | (Kabat) | NO: 27) | NO: 29) |
| | GFSLSTS | (Kabat) | (Kabat/ |
| | GM | WNDD | Chothia) |
| | (SEQ ID | (SEQ ID | |
| | NO: 25) | NO: 28) | |
| | (Chothia) | (Chothia) | |
| | GFSLSTS | | |
| | GMNVG | | |
| | (SEQ ID | | |
| | NO: 26) | | |
| | (Extended) | | |
| E10 | SYDVH | VIWSGGS | FSPWYL |
| | (SEQ ID | TGYNAIF | PSY |
| | NO: 37) | RS | (SEQ ID |
| | (Kabat) | (SEQ ID | NO: 42) |
| | GFSLTSY | NO: 40) | (Kabat/ |
| | (SEQ ID | (Kabat) | Chothia) |
| | NO: 38) | WSGGS | |
| | (Chothia) | (SEQ ID | |
| | GFSLTS | NO: 41) | |
| | YDVH | (Chothia) | |
| | (SEQ ID | | |
| | NO: 39) | | |
| | (Extended) | | |

In some embodiments, the anti-idiotype antibodies that specifically bind to anti-BCMA clones C29 or P5A2 and antigen binding molecules derived from C29 or P5A2, comprise a VL CDR 1, CDR2, and CDR3 of a VL sequence presented in Table 1b. In some embodiments, the VH CDR 1, CDR2, and CDR3 are selected from a CDR sequence presented in Table 1d.

TABLE 1d
Light Chain CDRs

| Clone | CDR L1 | CDR L2 | CDR L3 |
|---|---|---|---|
| H6 | KASEDI | GATRLET | QQYWS |
| | YNRLA | (SEQ ID | SSWT |
| | (SEQ ID | NO: | (SEQ ID |
| | NO: 12) | 13) | NO: 14) |
| | (Kabat & | (Kabat & | (Kabat & |
| | Chothia) | Chothia) | Chothia) |
| F3 | SVSSSI | GTSNL | QQWSSY |
| | SSSNLH | AS | PLT |
| | (SEQ ID | (SEQ ID | (SEQ ID |
| | NO: 30) | NO: 31) | NO: 32) |
| | (Kabat & | (Kabat & | (Kabat & |
| | Chothia) | Chothia) | Chothia) |
| E10 | SASSNV | STSNLAS | QQRSSY |
| | SYMH | (SEQ ID | PLT |
| | (SEQ ID | NO: 44) | (SEQ ID |
| | NO: 43) | (Kabat & | NO: 45) |
| | (Kabat & | Chothia) | (Kabat & |
| | Chothia) | | Chothia) |

In some of any such embodiments, the target antibody or antigen-binding fragment is within or including in the antigen-binding domain of the extracellular portion of a chimeric antigen receptor; and/or the anti-id antibody or antigen-binding fragment specifically binds the target antibody or antigen-binding fragment contained within or included in the antigen-binding domain of the extracellular portion of a CAR. In some embodiments, the target antibody or antigen-binding fragment is an scFv and the anti-id antibody or antigen-binding fragment specifically binds to an epitope in the scFv of the CAR.

In some of any such embodiments, the antibody or fragment specifically binds to a scFv derived from antibody C29 contained in the extracellular portion of a chimeric antigen receptor, optionally wherein the scFv derived from antibody C29 contains a heavy chain variable region set forth in SEQ ID NO: 15 and/or a light chain variable region set forth in SEQ ID NO: 16 and/or contains the sequence of amino acids set forth in SEQ ID NO: 1. In some of any such embodiments, the antibody or fragment specifically binds to a scFv derived from antibody P5A2 comprised in the extracellular portion of a chimeric antigen receptor, optionally wherein the scFv derived from antibody P5A2 contains a heavy chain variable region set forth in SEQ ID NO: 17 and/or a light chain variable region set forth in SEQ ID NO: 18; and/or contains the sequence of amino acids set forth in SEQ ID NO: 19.

In some of any such embodiments, the anti-idiotype antibody or antigen-binding fragment specifically binds to an epitope within or including all or a portion of a complementarity determining region (CDR) of the target antibody or antigen-binding fragment.

In some of any such embodiments, the anti-idiotype antibody or fragment is antagonist antibody of a CAR containing the target antibody or antigen-binding fragment. In some of any such embodiments, the antibody or fragment is an antagonist of a CAR containing the target antibody or antigen-binding fragment.

In some of any such embodiments, the anti-idiotype antibody or antigen-binding fragment thereof is humanized. In some of any such embodiments, the anti-idiotype antibody or antigen-binding fragment thereof is recombinant. In some of any such embodiments, the anti-idiotype antibody or antigen-binding fragment thereof is monoclonal.

In some of any such embodiments, the anti-idiotype antibody or antigen-binding fragment thereof is an antigen-binding fragment. In some aspects, the antigen-binding fragment is selected from among fragment antigen binding (Fab) fragments, F(ab′) 2 fragments, Fab′ fragments, Fv fragments, a single chain variable fragment (scFv) or a single domain antibody.

In some of any such embodiments, the anti-idiotype antibody or antigen-binding fragment thereof contains at least a portion of an immunoglobulin constant region. In some embodiments, the at least a portion of an immunoglobulin constant region contains an Fc region or a portion of the Fc containing the CH2 and CH3 domains. In some aspects, the constant region is derived from human IgG. In some of any such embodiments, the anti-idiotype antibody or antigen-binding fragment is an intact antibody or full-length antibody.

Provided herein is a vector containing the nucleic acid molecule according to any one of the embodiments described herein. Also provided herein is a cell containing the anti-idiotype antibody or antigen-binding fragment thereof according to any one of the embodiments described herein or the nucleic acid molecule according to any one of the embodiments described herein.

The disclosure encompasses modifications to the anti-id antibodies comprising the sequences shown in Tables 1a to 1d, including functionally equivalent anti-id antibodies having modifications which do not significantly affect their properties and variants which have enhanced or decreased activity and/or affinity. For example, the amino acid sequence can be mutated to obtain an anti-id antibody with the desired binding affinity to anti-BCMA clones C29 or P5A2 and antigen binding molecules derived from C29 or P5A2. Modification of polypeptides is routine practice in the art and thus need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or the use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antigen binding domain removed and a different residue inserted in its place. In some embodiments, sites of interest for substitutional mutagenesis include the hypervariable regions/CDRs, but FR alterations are also contemplated. Conservative substitutions are shown in Table 2 under the heading of “conservative substitutions”. If such substitutions result in a change in biological activity, then more substantial changes, denominated “exemplary substitutions” in Table 2, or as further described below in reference to amino acid classes, can be introduced and the products screened.

TABLE 2
Amino Acid Substitutions

| Original Residue | | |
|---|---|---|
| (naturally | | |
| occurring amino | Conservative | Exemplary |
| acid) | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr, Phe |
| Tyr (Y) | Phe | Trp; Phe; Thr; Ser |
| Val (V) | Leu | Ile; Leu; Met; Phe; Ala; |
| | | Norleucine |

### II. Methods of Making Anti-Id Antibodies

For cloning of polynucleotides, the vector can be introduced into a host cell (an isolated host cell) to allow replication of the vector itself and thereby amplify the copies of the polynucleotide contained therein. The cloning vectors can contain sequence components generally include, without limitation, an origin of replication, promoter sequences, transcription initiation sequences, enhancer sequences, and selectable markers. These elements can be selected as appropriate by a person of ordinary skill in the art. For example, the origin of replication can be selected to promote autonomous replication of the vector in the host cell.

In certain embodiments, the present disclosure provides isolated host cells containing the vector provided herein. The host cells containing the vector can be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescens, and Shigella, as well as Bacilli such as B. subtilis and B. licheniformis, Pseudomonas such as P. aeruginosa, and Streptomyces.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art. In a further embodiment, a mixture of different expression vectors can be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In one embodiment, the disclosure provides a method of evaluating genetically engineered cells expressing a CAR which targets a BCMA. In some embodiments the engineered cells are evaluated after thawing cryopreserved the immune cells.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion media can be any isotonic medium formulation, typically normal saline, Normosol™ R (Abbott) or Plasma-Lyte™ A (Baxter), but also 5% dextrose in water or Ringer's lactate can be utilized. The infusion medium can be supplemented with human serum albumin.

In some aspects, the anti-id antibodies of the present disclosure are used to quantify desired treatment amounts of cells in a composition of engineered T cells comprising a C29 or P5A2 derived CAR, e.g., an anti-BCMA CAR (such as a CAR comprising a C29 or P5A2 derived scFv) or a fragment thereof. In some embodiments, the desired treatment amount is generally at least 2 cells (for example, at least 1 CD8+ central memory T cell and at least 1 CD4+ helper T cell subset) or is more typically greater than 10² cells, and up to 10⁶, up to and including 10⁸ or 10⁹ cells and can be more than 10¹⁰ cells. The number of cells will depend upon the desired use for which the composition is intended, and the type of cells included therein. The density of the desired cells is typically greater than 10⁶ cells/ml and generally is greater than 10⁷ cells/ml, generally 10⁸ cells/ml or greater. A clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present disclosure, particularly since all the infused cells will be redirected to a particular target antigen (BCMA), lower numbers of cells, in the range of 10⁶/kilogram (10⁶-10¹¹ per patient) can be administered. CAR treatments can be administered multiple times at dosages within these ranges. The cells can be autologous, allogeneic, or heterologous to the patient undergoing therapy.

The CAR expressing cell populations of the present disclosure can be administered either alone, or as a pharmaceutical composition in combination with diluents and/or with other components such as IL-2 or other cytokines or cell populations. Pharmaceutical compositions of the present disclosure can comprise a CAR or TCR expressing cell population, such as T cells, as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for intravenous administration. The pharmaceutical compositions (solutions, suspensions or the like), can include one or more of the following: sterile diluents such as water for injection, saline solution such as physiological saline, Ringer's solution, isotonic sodium chloride, fixed oils such as synthetic mono- or diglycerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition can be sterile.

### III. Methods of Determining Numbers of Cells Expressing an Anti-BCMA CAR

The present disclosure provides a method to determine the number of cells present in a sample that are expressing an anti-BCMA CAR (e.g., a CAR comprising a C29- or P5A2-derived scFv) or a fragment thereof. For example, it can be desirable to determine the number of immune cells present in a sample obtained from a subject that are expressing an anti-BCMA CAR (e.g., a CAR comprising a C29- or P5A2-derived scFv) or a fragment thereof. Or it can be desirable to determine the number of cells transfected and expressing an anti-BCMA CAR (e.g., a CAR comprising a C29- or P5A2-derived scFv) or a fragment thereof, which can be used as a measure of the level of efficiency of the transfection. The disclosed method can be employed in these and other applications in which it is desirable to determine the number of cells present in a sample that are expressing a molecule of interest such as an anti-BCMA CAR (e.g., a CAR comprising a C29- or P5A2-derived scFv) or a fragment thereof.

Thus, a method of determining a number of cells presenting a molecule in a sample wherein the molecule comprises a polypeptide comprising an anti-BCMA CAR (e.g., a CAR comprising a C29- or P5A2-derived scFv) or a fragment thereof is provided.

In some embodiments, a sample comprising cells known or suspected to be expressing a molecule of interest comprising a polypeptide comprising an anti-BCMA CAR (e.g., a CAR comprising a C29- or P5A2-derived scFv) or a fragment thereof is provided.

The sample is then contacted with an antigen binding molecule that specifically binds the molecule of interest, under conditions that permit the formation of a binding complex comprising a cell present in the sample and the antigen binding molecule. The antigen binding molecule can be an antigen binding molecule (or fragment thereof) disclosed herein, e.g., in the Figures, Sequence Listing or the instant section of the disclosure. Any antigen binding molecule that specifically binds a polypeptide comprising an anti-BCMA CAR (e.g., a CAR comprising a C29- or P5A2-derived scFv) or a fragment thereof can be employed in the disclosed method. Multiple examples of suitable antigen binding molecules are provided herein, e.g., those having one or more of the CDRs shown in Tables 1c and 1d and described herein.

The cell can be of any type, and can be human or non-human (e.g., mouse, rat, rabbit, hamster, etc.). In some embodiments, the cell is an immune cell. An immune cell of the method can be any type of immune cell (e.g., B lymphocytes, monocytes, dendritic cells, Langerhans cells, keratinocytes, endothelial cells, astrocytes, fibroblasts, and oligodendrocytes). In some embodiments, the immune cells are T cells including T cytotoxic, T helper and Treg cells. In specific embodiments, the cells are T cells, which can be obtained as described herein and by methods known in the art. Any type of immune cell can be employed in this embodiment of the disclosed method, and the cell can be a human or non-human cell (including both prokaryotic and eukaryotic cells). Exemplary cells include, but are not limited to, immune cells such as T cells, tumor infiltrating lymphocytes (TILs), NK cells, TCR-expressing cells, dendritic cells, and NK-T cells. The T cells can be autologous, allogeneic, or heterologous. In additional embodiments, the cells are T cells presenting a CAR. The T cells can be CD4+ T cells or CD8+ T cells. When a T cell is employed in the disclosed methods, the T cell can be an in vivo T cell or an in vitro T cell. Moreover, the cells can be disposed in, or isolated from, any environment capable of maintaining the cells in a viable form, such as blood, tissue or any other sample obtained from a subject, cell culture media, tissue grown ex vivo, a suitable buffer, etc.

In some embodiments, the sample comprising an anti-BCMA CAR (e.g., a CAR comprising a C29- or P5A2-derived scFv) or a fragment thereof is contacted with an anti-id antibody disclosed herein that specifically binds a BCMA derived binding molecule. In some embodiments, the anti-id antibody comprises a detectable label. In some embodiments, the detectable label conjugated anti-id antibody is contacted with the sample expressing an anti-BCMA CAR (e.g., a CAR comprising a C29- or P5A2-derived scFv), under conditions that permit the formation of a binding complex comprising a cell present in the sample and the anti-id antibody. Any anti-id antibody that specifically binds an anti-BCMA CAR (e.g., a CAR comprising a C29- or P5A2-derived scFv) can be employed in the disclosed method. Multiple examples of suitable anti-id antibody are provided herein, e.g., those having one or more of the CDRs shown in Table 1c or 1d.

Any detectable label can be employed in the methods, as described herein, and suitable labels can be selected using a desired set of criteria. Examples of types of detectable labels include fluorescent labels (e.g., fluorescein, rhodamine, tetramethylrhodamine, eosin, erythrosin, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cas-cade Yellow and R-phycoerythrin (PE) (Molecular Probes), FITC, Rhodamine, and Texas Red (Pierce), Cy5, Cy5.5, Cy7 (Amersham Life Science). Suitable optical dyes, including fluoro-phores, are described in Johnson, Molecular Probes Handbook: A Guide to Fluorescent Probes and Labeling Techniques, 11^{th} Edition, Life Technologies, (2010), hereby expressly incorporated by reference, radiolabels (e.g., isotope markers such as ³H, ¹¹C, ¹⁴C ¹⁵N, ¹⁸F, ³⁵S, ⁶⁴CU, ⁹⁰Y, ⁹⁹Tc, ¹¹¹In, ¹²⁴I, ¹²⁵I, ¹³¹I), photochromic compounds, a Halo-tag, Atto dyes, Tracy dyes, proteinaceous fluorescent labels (e.g., proteinaceous fluorescent labels also include, but are not limited to, green fluorescent protein, including a Renilla, Ptilosarcus, or Aequorea species of GFP (Chalfie et al., (1994) Science 263:802-805), EGFP (Clon-tech Labs., Inc., Genbank Accession Number U55762), blue fluorescent protein (BFP, Quantum Biotechnologies, Inc; Stauber, (1998) Biotechniques 24:462-471; Heim et al., (1996) Curr. Biol. 6: 178-182), enhanced yellow fluorescent protein (Clontech Labs., Inc.), luciferase (Ichiki et al., (1993) J. Immunol. 150:5408-5417), magnetic labels (e.g., DYNABEADS), etc. Strategies for the labeling of proteins are well known in the art and can be employed in the disclosed method. See, e.g., Obermaier et al., (2015) Methods Mol Biol 1295:153-65; Strack (2016) Nature Methods 13:33; Site-Specific Protein Labeling: Methods and Protocols, (Gautier and Hinner, eds.) 2015, Springer. In some embodiments, the detectable label is a phycoerythrin (PE) or allophycocyanin (APC) fluorescent probe.

The label can be associated with the anti-id antibody at any position in the molecule, although it can be desirable to associate the label with the antibody at a position (or positions, if multiple labels are employed) at a point such that the binding properties of the molecule are not modified (unless such modified binding activity is desired). Any antigen binding molecule that specifically binds a BCMA binding molecule (or fragment thereof) can be employed, such as those disclosed herein, e.g., those having one or more of the CDRs shown in Table 1c or 1d.

The antigen binding molecule can be disposed on any surface, or no surface at all. For example, the antigen binding molecule can be present in a buffer and the buffer-antigen binding molecule can be contacted with the sample. Alternatively, the antigen binding molecule can be associated with a surface. Suitable surfaces include agarose beads, magnetic beads such as DYNABEADS®, or a plastic, glass or ceramic plate such as a welled plate, a bag such as a cell culture bag, etc. The surface can itself be disposed in another structure, such as a column.

Conditions that permit the formation of a binding complex will be dependent on a variety of factors, however generally aqueous buffers at physiological pH and ionic strength, such as in phosphate-buffered saline (PBS), will favor formation of binding complexes and are desirable in the disclosed method.

The number of cells present in a binding complex in the sample is determined. The specific method employed to determine the number of cells present in a binding complex will be dependent on the nature of the label selected. For example, FACS can be employed when a fluorescent label is selected; when an isotope label is selected mass spectrometry, NMR or other technique can be employed; magnetic-based cell sorting can be employed when a magnetic label is chosen; microscopy can also be employed. The output of these detection methods can be in the form of a number of cells or the output can be of a form that allows the calculation of the number of cells based on the output.

### IV. Methods of Determining the Presence or Absence of a C29 or P5A2 Derived Anti-BCMA CAR

In some embodiments, knowing whether a molecule comprising an anti-BCMA CAR (e.g., a CAR comprising a C29- or P5A2-derived scFv) or a fragment thereof, is present or absent from a sample is sufficient information. For example, it can be beneficial to know that such a molecule is being expressed, regardless of the level of expression. In other cases, it can be desirable to know if a purification process or step designed to remove such a molecule has been effective. Thus, the qualitative determination of the presence or absence of an anti-BCMA CAR (e.g., a CAR comprising a C29- or P5A2-derived scFv) or a fragment thereof, can be useful in multiple applications.

In some embodiments, a method of determining the presence or absence in a sample of a polypeptide comprising an anti-BCMA CAR (e.g., a CAR comprising a C29- or P5A2-derived scFv) or a fragment thereof, in a sample is provided.

In some embodiments, the method comprises providing a sample known or suspected to comprise a polypeptide comprising an anti-BCMACAR (e.g., a CAR comprising a C29- or P5A2-derived scFv) or a fragment thereof.

The disclosure provides an antigen binding molecule that specifically binds a polypeptide comprising an anti-BCMA CAR (e.g., a CAR comprising a C29- or P5A2-derived scFv) or a fragment thereof, which includes a detectable label. Suitable labels can be selected using a desired set of criteria. Examples of types of detectable labels include fluorescent labels (e.g., fluorescein, rhodamine, tetramethylrhodamine, eosin, erythrosin, coumarin, methyl-coumarins, pyrene, Malachite green, stilbene, Lucifer Yellow, Cascade Blue, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 647, Alexa Fluor 660, Alexa Fluor 680), Cascade Blue, Cas-cade Yellow and R-phycoerythrin (PE) (Molecular Probes), FITC, Rhodamine, and Texas Red (Pierce), Cy5, Cy5.5, Cy7 (Amersham Life Science)). Suitable optical dyes, including fluorophores, are described in Johnson, Molecular Probes Handbook: A Guide to Fluorescent Probes and Labeling Techniques, 11^{th} Edition, Life Technologies, (2010), hereby expressly incorporated by reference, radiolabels (e.g., isotope markers such as ³H, ¹¹C, ¹⁴C, ¹⁵N, ¹⁸F, ³⁵S, ⁶⁴CU, ⁹⁰Y, ⁹⁹Tc, ¹¹¹In, ¹²⁴I, ¹²⁵I, ¹³¹I). Photochromic compounds, a Halo-tag, Atto dyes, Tracy dyes, proteinaceous fluorescent labels (e.g., proteinaceous fluorescent labels also include, but are not limited to, green fluorescent protein, including a Renilla, Ptilosarcus, or Aequorea species of GFP (Chalfie et al., (1994) Science 263:802-805), EGFP (Clon-tech Labs, Inc., Genbank Accession Number U55762), blue fluorescent protein (BFP, Quantum Biotechnologies, Inc.; Stauber, (1998) Biotechniques 24:462-471; Heim et al., (1996) Curr. Biol. 6: 178-182), enhanced yellow fluorescent protein (Clontech Labs, Inc.), luciferase (Ichiki et al., (1993) J. Immunol. 150:5408-5417), magnetic labels (e.g., DYNABEADS®), etc. can also be employed. Strategies for the labeling of proteins are well known in the art and can be employed in the disclosed methods. The label can be associated with the antigen binding molecule at any position in the molecule, although it can be desirable to associate the label with the molecule at a position (or positions, if multiple labels are employed) at a point such that the binding properties of the molecule are not modified (unless such modified binding activity is desired). Any antigen binding molecule that specifically binds a polypeptide comprising an anti-BCMA CAR (e.g., a CAR comprising a C29- or P5A2-derived scFv) or a fragment thereof can be employed, such as those disclosed herein, e.g., those having one or more of the CDRs described in Tables 1c and 1d.

Next, the sample is contacted with the antigen binding molecule under conditions that permit the formation of a binding complex comprising a cell present in the sample and the antigen binding molecule.

The sample is contacted with the antigen binding molecule, under conditions that permit the formation of a binding complex between a polypeptide comprising an anti-BCMA CAR (e.g., a CAR comprising a C29- or P5A2-derived scFv) or a fragment thereof and the antigen binding molecule. Conditions that permit the formation of a binding complex will be dependent on a variety of factors. Since the component parts of a binding complex can be disposed on surfaces as described herein, formed binding complexes can also be disposed on surfaces.

At this stage, no binding complexes can have formed, or a plurality of binding complexes comprising one or more antigen binding molecules bound to a polypeptide comprising an anti-BCMA CAR (e.g., a CAR comprising a C29- or P5A2-derived scFv) or a fragment thereof can have formed. Unbound molecules comprising an anti-BCMA CAR (e.g., a CAR comprising a C29- or P5A2-derived scFv) or a fragment thereof and/or unbound antigen binding molecules can also be present in the local environment of any formed binding complexes.

Any molecules not part of a binding complex are then separated from any formed binding complexes. The method of the removal will depend on the structure and/or local environment of the binding complexes. For example, if the antigen binding molecule is disposed on a bead, plate or bag the unbound components of the reaction mixture can be washed away using a solution that leaves formed binding complexes intact. In some embodiments, separation of the binding complex is not required for detection.

The solution used to induce the formation of binding complexes can be used, for example, as a wash solution to remove unbound components. Any suitable buffer or solution that does not disrupt formed binding complexes can also be used. Typically, buffers having high salt concentrations, non-physiological pH, containing chaotropes or denaturants, should be avoided when performing this step of the method.

The presence or absence of a binding complex, which will comprise a polypeptide comprising an anti-BCMA CAR (e.g., a CAR comprising a C29- or P5A2-derived scFv) or a fragment thereof and an antigen binding molecule, can be detected. The specific method employed to detect the presence or absence of a binding complex will typically be dependent on the nature of the label selected. In some embodiments, the detection method is by colorimetric assay. The result of the method is a qualitative assessment of the presence or absence of the antigen binding molecule comprising the detectable label, and thus, the presence or absence of its binding partner, a polypeptide comprising an anti-BCMA CAR (e.g., a CAR comprising a C29- or P5A2-derived scFv) or a fragment thereof.

As is the case with the disclosed methods, the polypeptide comprising an anti-BCMA CAR (e.g., a CAR comprising a C29- or P5A2-derived scFv) or a fragment thereof can be disposed in any environment. In some embodiments, the polypeptide comprising an anti-BCMA CAR (e.g., a CAR comprising a C29- or P5A2-derived scFv) or a fragment thereof is expressed on the surface of a cell. In this embodiment, the cell can be of any type, and can be human or non-human (e.g., mouse, rat, rabbit, hamster, etc.). In some embodiments, the cell is an immune cell. An immune cell of the method can be any type of immune cell (e.g., B lymphocytes, monocytes, dendritic cells, Langerhans cells, keratinocytes, endothelial cells, astrocytes, fibroblasts, and oligodendrocytes). T cells (including T cytotoxic, T helper and Treg cells) are especially suitable. In specific embodiments, the cells are T cells, which can be obtained as described herein and by methods known in the art. Any type of immune cell can be employed in this embodiment of the disclosed method, and the cell can be a human or non-human cell. Exemplary cells include, but are not limited to, immune cells such as T cells, tumor infiltrating lymphocytes (TILs), NK cells, dendritic cells, and NK-T cells. The T cells can be autologous, allogeneic, or heterologous. In additional embodiments, the cells are T cells presenting a TCR. The T cells can be CD4+ T cells or CD8+ T cells. When a T cell is employed in the disclosed methods, the T cell can be an in vivo T cell or an in vitro T cell. Further, cells can be derived from a stem cell, such as an iPSC cell, cord blood cell, or mesenchymal stem cell.

In some embodiments, the cell can be disposed in, or isolated from, any environment capable of maintaining the cell in a viable form, such as blood, tissue or any other sample obtained from a subject, cell culture media, tissue grown ex vivo, a suitable buffer, etc. In some embodiments, the cell is in a formalin-fixed sample. In some embodiments, the sample is a formalin-fixed paraffin embedded tissue (FFPE).

### V. Methods and Uses of the Antibodies

In some embodiments, provided herein are methods involving the use of one or more anti-idiotype antibodies. In some aspects, provided herein are methods for measuring or detecting a target antibody, such as a CAR or a cell expressing a CAR, and methods for modifying the activity of the target antibody, such as the activity of a CAR or the activity of a cell expressing a CAR. In certain embodiments, the one or more anti-idiotype antibodies bind, detect, identify, and/or quantify the CAR and/or cells expressing the CAR. In some embodiments, the methods provided herein provide one or more steps of contacting and/or incubating the one or more anti-idiotype antibodies with a cell or a sample containing or thought to be containing cells that express a chimeric antigen receptor (CAR). In some embodiments, the anti-idiotype antibody is treated, incubated, and/or contacted with the composition or sample under conditions that allow for the formation of a complex between the anti-idiotype antibody and the target antibody, e.g., the CAR. In some aspects, the complex may be utilized for the purposes of detecting, isolating, and/or measuring the CAR. In some embodiments, the formation of the complex modifies the activity of the target antibody, e.g., the CAR, such as by stimulating receptor signaling activity, or in some embodiments, antagonizing the activity of the target antibody, e.g., the CAR, by preventing the association of the CAR with an antigen.

A. Detection/Isolation Methods

In some embodiments, there are provided methods involving use of one or more of the anti-idiotype antibodies, and/or molecules (such as conjugates and complexes) containing one or more of such anti-idiotype antibodies, for detecting, binding, and/or isolating an antibody, e.g., a target antibody. In certain embodiments, the methods provide one or more steps of contacting, incubating, and/or exposing the one or more anti-idiotype antibodies to a sample and/or composition. In some embodiments, the sample and/or composition has, is likely to have, and/or is suspected of having a target antibody and/or antigen binding fragment thereof that is bound by and/or recognized by the one or more anti-idiotype antibodies. In certain embodiments, the antibody or antigen binding fragment thereof that is bound by or recognized by the one or more anti-idiotype antibodies contains one or more fusion domains and/or is a fusion protein. In certain embodiments, the target antibody and/or antigen binding fragment thereof is a CAR. In certain embodiments, the anti-idiotype antibody, binds to and/or recognizes an anti-BCMA antibody (e.g., antibody C29 or P5A2), or an antigen-binding fragment thereof, including a chimeric molecule or conjugate including a CAR, containing such anti-BCMA antibody (e.g., antibody fragment).

The methods in some embodiments include incubating, treating, and/or contacting a sample and/or a composition containing or suspected of containing the target antibody with the anti-idiotype antibody. In certain embodiments, the incubating is under conditions permissive for binding of the anti-idiotype antibody to the target antibody present in the composition, for example to form a complex containing the anti-idiotype antibody and the target antibody.

In some embodiments, the sample and/or composition contains or is suspected of containing the target antibody, e.g., a CAR. In certain embodiments, the sample and/or composition contains or is suspected of containing cells that express the target antibody, e.g., a CAR. In certain embodiments, the sample is a biological sample. In particular embodiments, the sample is a serum sample or a blood sample. In some embodiments, the biological sample contains one or more immune cells. In some embodiments, the biological sample is or is derived from a tissue, such as connective tissue, muscle tissue, nervous tissue, or epithelial tissue. In particular embodiments, the biological sample is taken, collected, and/or obtained from a human subject. In certain embodiments, the sample contains cells that are live and/or intact. In some embodiments, the sample is or contains a homogenate and/or cells that have been disrupted and/or lysed. In some embodiments, the biological sample contains proteins and/or antibodies that have been isolated from blood, serum, and/or a tissue.

In particular embodiments, the anti-idiotype antibody forms or is capable of forming a complex with a target antibody, e.g., a CAR. In particular embodiments, the complex is detected, measured, quantified, and/or assessed, for example, to allow for the detection, identification, measurement, and/or quantification of the target antibody, for example in a composition or a sample. In certain embodiments, the methods include detecting whether a complex is formed between the anti-idiotype antibody and the target antibody in the sample, and/or detecting the presence or absence or level of such binding. In some embodiments, the complex contains a detectable label. In particular embodiments, the anti-idiotype antibody is an immunoconjugate that contains a detectable label. In certain embodiments, the anti-idiotype antibody contains, is conjugated with, bound to, and/or attached to the detectable label. In some embodiments, the complex contains an antibody that binds to and/or recognizes the anti-idiotype antibody, e.g., a secondary antibody, that in conjugated with, bound to, and/or attached to a detectable label.

In some embodiments, methods for detecting, quantifying, detecting, and/or assessing a target antibody, for example in a sample or composition, includes detecting a complex of the target antibody and the anti-idiotype antibody. In some embodiments, the complex contains a detectable label. In certain embodiments, the complex is probed and/or contacted with a detectable label. In some embodiments, the complex is detected by any suitable method or means, such as but not limited to flow cytometry, immunocytochemistry, immunohistochemistry, western blot analysis, and ELISA.

In some embodiments, the target antibody or antigen-binding fragment is bound to a cell or expressed on the surface of a cell. In particular embodiments, target antibody, e.g., the CAR is not bound or contained within a cell, for example, in some embodiments, the target antibody is secreted. In certain embodiments, the antibody has been detached, removed, and/or lysed from the surface of a cell.

In some embodiments, the target antibody is an anti-BCMA antibody. In some embodiments, the target antibody is or is derived from antibody C29 or an antigen-binding fragment thereof. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody is or is derived from antibody P5A2 or an antigen-binding fragment thereof. In some embodiments, the target antibody or antigen-binding fragment thereof comprises a heavy chain variable region set forth in SEQ ID NO: 17 and/or a light chain variable region set forth in SEQ ID NO: 19.

In some embodiments, there is provided a method of detecting a target antibody, such as antibody C29, or an antigen-binding fragment thereof (and/or chimeric molecules comprising such antibody, e.g., antibody fragment, such as a CAR), comprising contacting a composition comprising the target antibody or antigen-binding fragment with an anti-idiotype antibody or antigen-binding fragment thereof or an anti-idiotype antibody immunoconjugate described herein, and detecting the anti-idiotype antibody bound to the target antibody or antigen-binding fragment. In some embodiments, the method further includes detecting whether a complex is formed between the anti-idiotype antibody and the target antibody in the composition, such as detecting the presence or absence or level of such binding. In some embodiments, the target antibody or antigen-binding fragment is bound to a cell or expressed on the surface of a cell and the detecting comprises detecting cells bound with the anti-idiotype antibody. In some embodiments, the anti-idiotype antibody or antigen-binding fragment thereof is directly or indirectly labeled for detection. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

In some embodiments, there is provided a method of isolating a target antibody, such as antibody P5A2, or an antigen-binding fragment thereof (and/or chimeric molecules comprising such antibody, e.g., antibody fragment, such as a CAR), comprising contacting a composition and/or a sample containing or suspected of containing the target antibody or antigen-binding fragment with an anti-idiotype antibody or antigen-binding fragment thereof or an anti-idiotype antibody immunoconjugate described herein, and isolating complexes comprising the anti-idiotype antibody bound to the target antibody or antigen-binding fragment. In certain embodiments, the target antibody is isolated with an anti-idiotype antibody described in section I. In some embodiments, the target antibody or antigen-binding fragment is bound to a cell or expressed on the surface of a cell and the isolating comprises isolating cells bound with the anti-idiotype antibody. In some embodiments, the complexes comprising the anti-idiotype antibody are isolated by affinity-based separation. In some embodiments, the affinity-based separation is selected from the group consisting of immunoaffinity-based separation, magnetic-based separation, and affinity chromatography. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

In certain embodiments, the methods for detecting a target antibody with an anti-idiotype antibody described herein are used to assess the target antibody in a subject. For example, in some embodiments, provided herein are methods of use for the anti-idiotype antibody for assessing, measuring, and/or quantifying the in vivo pharmacokinetics, expansion, and/or persistence of a CAR expressing cells of a therapeutic cell composition. In some embodiments, the in vivo pharmacokinetics, expansion, and/or persistence of the cells, and/or changes in cell phenotypes or functional activity of cells, such as CAR expressing cells administered for immunotherapy, e.g. CAR-T cell therapy, in the methods provided herein, can be measured with the anti-idiotype antibodies provided herein. In some embodiments, the pharmacokinetics, expansion, and/or persistence of the CAR expressing cells are measured, assessed by detecting the presence and/or amount of cells expressing the CAR in the subject and/or in sample obtained from the subject following the administration of the therapeutic cell composition during and/or after the administration of the therapy with an anti-idiotype antibody provided herein.

In some aspects, the anti-idiotype antibody is used with flow cytometry to assess the quantity of cells expressing the recombinant receptor (e.g., CAR-expressing cells administered for T cell based therapy) in the blood or serum or organ or tissue sample (e.g., disease site, e.g., tumor sample) of the subject. In some aspects, persistence is quantified as the number of CAR-expressing cells per microliter of the sample, e.g., of blood or serum, or per total number of peripheral blood mononuclear cells (PBMCs) or white blood cells or T cells per microliter of the sample. In certain aspects, expansion is quantified as the increase in the number of CAR-expressing cells per microliter between samples, e.g., of blood or serum, or per total number of peripheral blood mononuclear cells (PBMCs) or white blood cells or T cells per microliter of the samples over time. In some embodiments, the pharmacokinetics, expansion, and/or persistence are measured or assessed by detecting the amount of CAR expressing cells in the subject and/or in samples collected from the subject at multiple time points.

In some embodiments, there is provided a method of selecting cells expressing a CAR comprising a target antibody, such as antibody C29 or P5A2, or an antigen-binding fragment thereof, comprising contacting a population of cells comprising cells expressing the CAR with an anti-idiotype antibody or antigen-binding fragment thereof described herein, and selecting cells bound with the anti-idiotype antibody. In some embodiments, the cells bound with the anti-idiotype antibody are selected by affinity-based separation. In some embodiments, the affinity-based separation is selected from the group consisting of immunoaffinity-based separation, flow cytometry, magnetic-based separation, and affinity chromatography. In some embodiments, the anti-idiotype antibody or antigen-binding fragment thereof or anti-idiotype antibody immunoconjugate is reversibly bound or immobilized to a support or a stationary phase. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

In some embodiments, there is provided a method of validating a CAR comprising a target antibody, such as antibody C29 or P5A2, or an antigen-binding fragment thereof, comprising a) incubating a sample comprising T cells transduced with the CAR with an anti-idiotype antibody or antigen-binding fragment thereof targeting the CAR; b) determining the percent of cells bound with the anti-idiotype antibody or antigen-binding fragment thereof; and c) validating the CAR based on the percent of anti-idiotype antibody-bound T cells. In some embodiments, the anti-idiotype antibody is labeled, and anti-idiotype antibody-bound T cells are assayed by flow cytometry. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

Also provided are methods involving use of the provided anti-idiotype antibodies, and molecules (such as conjugates and complexes) containing one or more of such anti-idiotype antibodies, for informing treatment decisions in an individual, such as by the detection of CARs recognized by the anti-idiotype antibody, such as CARs comprising a target antibody, such as an anti-BCMA antibody (e.g., antibody C29 or P5A2), or an antigen-binding fragment thereof. In some embodiments, the methods are for informing treatment decisions in an individual in association with a therapy comprising administration of CAR T cells, such as anti-BCMA CAR T cells. The methods in some embodiments include incubating and/or probing a biological sample with the anti-idiotype antibody and/or administering the anti-idiotype antibody to the individual. In certain embodiments, a biological sample includes a cell or tissue or portion thereof, such as tumor or cancer tissue or biopsy or section thereof. In certain embodiments, the incubating is under conditions permissive for binding of the anti-idiotype antibody to CARs present in the sample. In some embodiments, the methods further include detecting whether a complex is formed between the anti-idiotype antibody and CARs in the sample, such as detecting the presence or absence or level of such binding. Such a method may be an in vitro or in vivo method.

B. Use in Cell Stimulation

In some embodiments, the provided anti-idiotype antibodies or antigen-binding fragments thereof are agonists and/or exhibit specific activity to stimulate cells expressing a target antibody including conjugates or chimeric receptors containing the same, such as an anti-BCMA antibody (e.g., antibody C29 or P5A2), or an antigen-binding fragment thereof. In some embodiments, provided are methods involving use of the provided anti-idiotype antibodies, and molecules (such as conjugates and complexes) containing one or more of such anti-idiotype antibodies, for stimulation or activation of CAR-expressing or other chimeric receptor-expressing cells, such as T cells. In some aspects, the CAR or other receptor comprises the target antibody, such as an anti-BCMA antibody (e.g., antibody C29 or P5A2), or an antigen-binding fragment thereof.

In some embodiments, the methods can be used in connection with methods of preparing genetically engineered T cells, such as in methods of expanding genetically engineered T cells or other cells into which a nucleic acid molecule encoding the chimeric receptor such as the CAR comprising the target antibody has been introduced, e.g., by transfection, transduction, or a non-viral means of nucleic acid transfer, such as transposon-based approaches. In some aspects, the target antibody is an anti-BCMA antibody (e.g., antibody C29 or P5A2), or an antigen-binding fragment thereof. In particular embodiments, the target antibody is or contains a CAR, e.g., an anti-BCMA CAR. In particular embodiments, the anti-BCMA CAR contains an scFv that is from and/or is derived from an anti-BCMA antibody such as antibody C29 or P5A2.

The methods in some embodiments include incubating a sample comprising T cells transduced with a CAR with the anti-idiotype antibody. In certain embodiments, the methods further include detecting whether the CAR T cells are activated or stimulated, such as by assessing the viability, proliferation, and/or expression of activation markers in the CAR T cells. In some embodiments, the target antibody is an anti-BCMA antibody. In some embodiments, the target antibody is or is derived from antibody C29 or P5A2 or an antigen-binding fragment thereof. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

In some embodiments, there is provided a method of simulating cells, comprising incubating an input composition comprising cells expressing a CAR comprising a target antibody, such as antibody C29 or P5A2, or an antigen-binding fragment thereof, with an anti-idiotype antibody or antigen-binding fragment thereof described herein, thereby generating an output composition comprising stimulated cells. In some embodiments, the incubation is performed under conditions in which the anti-idiotype antibody or antigen-binding fragment thereof binds to the CAR, thereby inducing or modulating a signal in one or more cells in the input composition. In some embodiments, the cells comprise T cells. In some embodiments, the T cells comprise CD4+ and/or CD8+ T cells.

In certain embodiments, the anti-idiotype antibody is administered to a subject, such as a subject who has previously been administered a therapeutic cell composition containing CAR expressing cells. In some embodiments, administering the anti-idiotype antibody to a subject promotes re-expansion of the CAR expressing cells in the subject, which, in some cases, may reach or exceed the initial peak level of expansion prior to the administration of the anti-idiotype antibody. In some embodiments, the anti-idiotype antibody is administered to modulate expansion and/or persistence of the CAR expressing cells at times when the levels of the CAR expressing cells have declined or are not detectable. In some embodiments, CAR expressing cells that re re-expanded by the anti-idiotype antibody exhibit increased potency in a subject to which it is administered, for example, as compared to the potency prior to administration of the anti-idiotype antibody.

In some embodiments, there is provided a method of producing a cell composition, comprising introducing into cells a nucleic acid molecule encoding a CAR, thereby generating an input composition, and incubating the input composition with an anti-idiotype antibody or antigen-binding fragment thereof specific for the antigen-binding domain of the CAR, thereby producing the cell composition. In some embodiments, the CAR comprises a target antibody or antigen-binding fragment thereof that specifically binds to BCMA. In some embodiments, the target antibody is antibody C29 or P5A2 or an antigen-binding fragment thereof. In some embodiments, the anti-idiotype antibody or antigen-binding fragment thereof is an anti-idiotype antibody or antigen-binding fragment thereof described herein. In some embodiments, the anti-idiotype antibody or antigen-binding fragment thereof is an agonist of the CAR. In some embodiments, the introducing comprises introducing the nucleic acid molecule into the cells by viral transduction, transposition, electroporation, or chemical transfection. In some embodiments, the introducing comprises introducing the nucleic acid molecule in the cells by transduction with a retroviral vector comprising the nucleic acid molecule, by transduction with a lentiviral vector comprising the nucleic acid molecule, by transposition with a transposon comprising the nucleic acid molecule, or by electroporation or transfection of a vector comprising the nucleic acid molecule.

C. Use in Cell Inactivation/Depletion

In some embodiments, the provided anti-idiotype antibodies or antigen-binding fragments thereof are antagonists and/or exhibit specific activity to inhibit, ablate, and/or deplete (for example, kill via antibody-dependent cell-mediated cytotoxicity, ADCC) cells expressing a target antibody, such as an anti-BCMA antibody (e.g., antibody C29 or P5A2), or an antigen-binding fragment thereof. Also provided are methods involving use of the provided anti-idiotype antibodies, and molecules (such as conjugates and complexes) containing one or more of such anti-idiotype antibodies, for inactivation, ablation, and/or depletion of CAR T cells, wherein the CAR comprises a target antibody, such as an anti-BCMAantibody (e.g., antibody C29 or P5A2), or an antigen-binding fragment thereof.

The methods in some embodiments include treating, contacting, and/or incubating a composition and/or a sample comprising T cells transduced with a CAR with the anti-idiotype antibody. In certain embodiments, the methods further include detecting whether the CAR T cells are inactivated, such as by assessing the viability, proliferation, and/or expression of activation markers in the CAR T cells. In some embodiments, the methods are in association with a therapy comprising administration of CAR T cells. The methods in some embodiments include administering the anti-idiotype antibody to an individual. In one embodiment, an anti-idiotype antibody or conjugate is used to ablate and/or deplete (such as kill) CAR T cells in an individual. In some embodiments, the target antibody is an anti-BCMA antibody. In some embodiments, the target antibody is or is derived from antibody C29 or P5A2 or an antigen-binding fragment thereof. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

In some embodiments, the anti-idiotype antibody is administered to deplete, reduce, and/or decrease the number of CAR expressing cells in a subject. In particular embodiments, administration of the anti-idiotype antibody depletes, reduces, and/or decreases the amount of CAR expressing cells, e.g., circulating CAR-T cells, by at least 25%, at least 50%, at least 60%, at least 70%, at least 80%, at least 90%, at least 95%, at least 99%, at least 99.9%, 100% or about 100%. In certain embodiments, the depletion, reduction, and/or decrease is in relation to an amount of CAR expressing cells in the subject prior to the administration of the anti-idiotype antibody. In particular embodiments, the depletion, reduction, and/or decrease is in relation to an amount of CAR expressing cells in a subject that is not administered the anti-idiotype antibody. In some embodiments, CAR expressing cells are not detectable in the subject following administration of the anti-idiotype antibody. In particular embodiments, the anti-idiotype antibody is a human or humanized antibody.

In some embodiments, there is provided a method of inactivating CAR T cells, wherein the CAR comprises a target antibody, such as antibody C29 or P5A2, or an antigen-binding fragment thereof, comprising incubating a sample comprising the CAR T cells with an antagonistic anti-idiotype antibody or antigen-binding fragment thereof targeting the CAR, thereby inactivating the CAR T cells in the sample. In some embodiments, the anti-idiotype antibody is used in an amount sufficient to attenuate the activation of the CAR T cells in the sample. In some embodiments, the anti-idiotype antibody is used in an amount sufficient to substantially inactivate the CAR T cells in the sample. In some embodiments, incubation with the anti-idiotype antibody results in ablation and/or depletion of CAR T cells in the sample. In some embodiments, the anti-idiotype antibody is used in an amount sufficient to result in clearance of the CAR T cells in the sample. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

In some embodiments, there is provided a method of adjusting a CART cell therapy in an individual, wherein the CAR comprises a target antibody, such as antibody C29 or P5A2, or an antigen-binding fragment thereof, comprising administering an anti-idiotype antibody immunoconjugate targeting the CAR to the individual, wherein the anti-idiotype antibody immunoconjugate comprises a cytotoxic agent. In some embodiments, the anti-idiotype antibody immunoconjugate is administered in an amount sufficient to attenuate the CAR T cell therapy in the individual. In some embodiments, the anti-idiotype antibody immunoconjugate is administered in an amount sufficient to substantially stop the CAR T cell therapy in the individual. In some embodiments, the anti-idiotype antibody immunoconjugate is administered in an amount sufficient to result in clearance of the CAR T cells in the individual. In some embodiments, the cytotoxic agent is selected from the group consisting of chemotherapeutic agents or drugs, growth inhibitory agents, toxins (e.g., protein toxins, enzymatically active toxins of bacterial, fungal, plant, or animal origin, or fragments thereof), and radioactive isotopes. In some embodiments, the target antibody or antigen-binding fragment thereof comprises a heavy chain variable region set forth in SEQ ID NO: 15 and/or a light chain variable region set forth in SEQ ID NO: 16. In some embodiments, the target antibody or antigen-binding fragment thereof comprises a heavy chain variable region set forth in SEQ ID NO: 17 and/or a light chain variable region set forth in SEQ ID NO: 18.

D. Use in Binding Assay or Method

Provided herein are methods for assessing the presence or absence of a molecule in a sample that binds to a chimeric antigen receptor (CAR), such as the extracellular domain of a CAR or to a portion thereof containing the antigen-binding domain. In some embodiments, the methods can be used to assess the presence or absence of a humoral response or antibody response in a subject to an administered cell therapy comprising a chimeric antigen receptor (CAR). In some embodiments, the chimeric antigen receptor comprises a target antibody that is antibody P5A2 or an antigen-binding fragment thereof. In some embodiments, the chimeric antigen receptor comprises a target antibody that is antibody C29 or an antigen-binding fragment thereof. In some embodiments, an anti-idiotype antibody or antigen-binding fragment thereof specific to the extracellular domain of the CAR, such as any described herein, can be used as a positive control in the method.

In some embodiments, there are provided methods that involve contacting or incubating a binding reagent with a sample from a subject having been administered a cell therapy comprising cell engineered with a chimeric antigen receptor in which the binding reagent is a protein that includes the extracellular domain of the CAR or a portion thereof containing the target antibody or the antigen-binding fragment thereof. In some embodiments, the methods further include detecting whether a complex is formed between the binding reagent and a molecule, e.g. binding molecule, such as an antibody, present in the sample, and/or detecting the presence or absence or level of such binding. In certain embodiments, the contacting or incubating is under conditions permissive for binding of the binding reagent to a molecule present in the sample from the subject. In certain aspects, the method can be further carried out on a positive control sample containing an anti-idiotypic antibody or antigen-binding fragment thereof specific for the CAR, such as any as described. In some embodiments, determining the presence, absence or level of binding of the molecule to the binding reagent can include comparison of the binding or detection to the binding or detection of the positive control sample to the binding reagent.

In some embodiments, the methods include detecting whether a complex is formed between the binding reagent and a molecule, e.g. binding molecule, such as an antibody, present in the sample, and/or detecting the presence or absence or level of such binding. In certain embodiments, the contacting or incubating is under conditions permissive for binding of the binding reagent to a molecule present in the sample from the subject. In some aspects, the complex is detected by an immunoassay, optionally a sandwich or bridge assay. For examples, the immunoassay is an enzyme-linked immunosorbent assay (ELISA), chemiluminescent, electrochemiluminescent, surface plasmon resonance (SPR)-based biosensor (e.g., BIAcore), flow cytometry, or Western blot. In some embodiments, the immunoassay is or includes meso scale discovery.

In some aspects, the immunoassay is a sandwich assay or a bridge assay. In a sandwich or bridge assay, the binding reagent is a first binding reagent and detecting the presence or absence of a molecule or a complex comprising a molecule includes contacting the complex formed between the first binding reagent and molecule with a second binding reagent in which the second binding reagent is an agent that is able to bind to the same or similar molecule as the first binding reagent. In some embodiments, the second binding reagent comprises the extracellular domain of the CAR or a portion thereof. In some aspects, the extracellular domain of the CAR or portion thereof of the first binding agent and the second binding agent is the same or substantially the same.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control.

The foregoing written specification is considered to be sufficient to enable one skilled in the art to practice the disclosure. The foregoing description and Examples that follow detail certain embodiments of the disclosure and describe the best mode contemplated by the inventors. It will be appreciated, however, that no matter how detailed the foregoing can appear in text, the disclosure can be practiced in many ways and the disclosure should be construed in accordance with the appended claims and any equivalents thereof.

### EXAMPLES

### Example 1: Generation of Anti-Idiotypic Antibodies to Anti-Human BCMA Antibody Clone C29

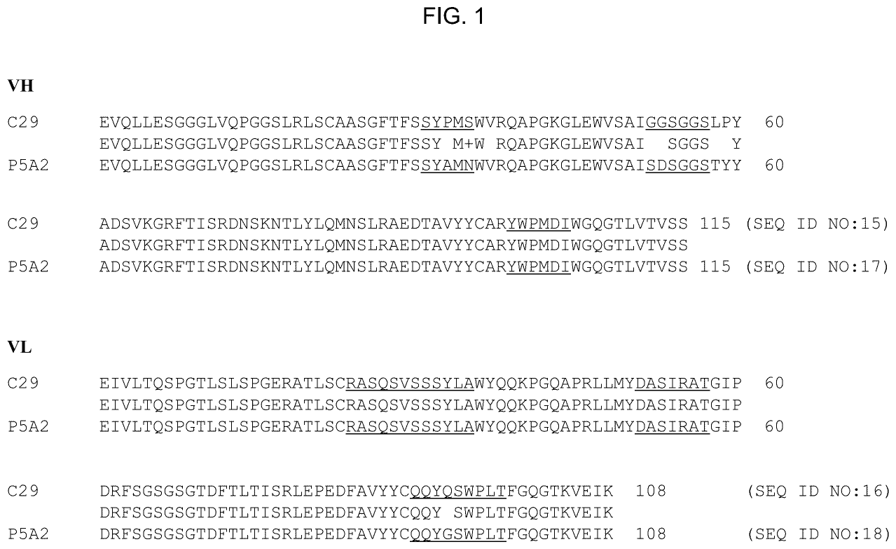

Balb/c mice were immunized with an anti-BCMA antibody clone C29-human Fab-8×His (HHHHHHHH-SEQ ID NO: 46) fusion protein (Combo29DI_Fab). Hybridomas were generated, cloned, and the secreted antibodies were screened for binding specificity to C29. Antibody clones were identified and selected based on their ability to specifically bind the C29-derived scFv. Anti-id clone E2008 was isolated and several E2008-derived subclones were purified for further analysis. Because the anti-BCMA antibody C29 shares high sequence identity with anti-BCMA antibody P5A2 (see FIG. 1), the anti-id antibody clones were also screened for specific binding to P5A2. Three anti-id clones E2008.51H1.H6 (H6), 54E4.F3 (F3) and 87B12.E10 (E10) showed promising activities and were further sequenced. Either the antibodies produced from hybridoma cell lines or antibodies recombinantly produced were tested. The anti-id clones E2008.51H1.H6 (H6) and 87B12.E10 (E10) were analyzed below.

### Example 2: Anti-Idiotypic Antibody H6 and E10 Against Anti-Human BCMA Antibody Clone C29 or P5A2

E10 purified from the hybridoma cell line supernatant was analyzed by Biacore assay against the antigen anti-BCMA clone C29 Fab. The Biacore assay was run, for example, in 10 mM HEPES buffer, with 150 mM NaCl and 0.05% Tween20 at pH 7.4 and 1 mg/mL BSA at 25° C. E10 did not bind to a Flt3 Fab that was used as a framework control (data not shown).

Similarly, the binding of clone H6, either purified from the hybridoma cell line or produced recombinantly, to the anti-BCMA scFv P5A2, which shares high sequence identity to C29 (see FIG. 1), was analyzed by Biacore. The results are shown in the table below.

TABLE 3
Binding specificity of anti-BCMA anti-id antibody E10 and H6

| Sample ID | kₐ (1/Ms) | k_{d} (1/s) | t_{1/2} (min) | K_{d} (nM) |
|---|---|---|---|---|
| E10 purified Ab from | 1.48E+06 | 1.57E−04 | 74 | 0.11 (nM) |
| hybridoma cell line | | | | |
| binding to C29 Fab | | | | |
| H6 purified Ab from | 1.86E+06 | 6.93E−03 | 1.67 | 3.73 (nM) |
| hybridoma cell line | | | | |
| binding to P5A2 scFv | | | | |
| H6 recombinant Ab | 1.90E+06 | 7.05E−03 | 1.64 | 3.71 (nM) |
| binding to P5A2 scFv | | | | |

### Example 3: Flow Analysis of Anti-BCMA Anti-Id Antibody H6

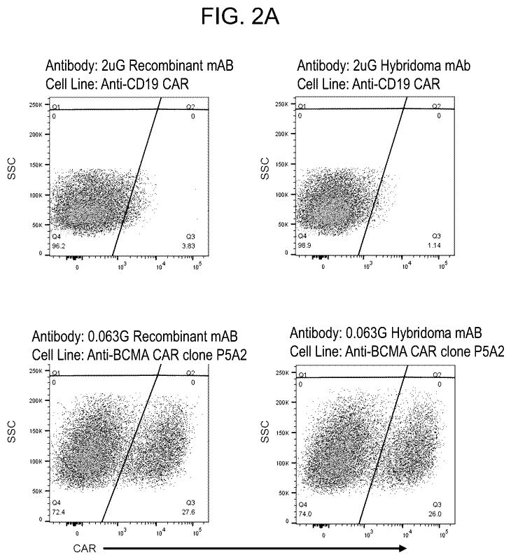

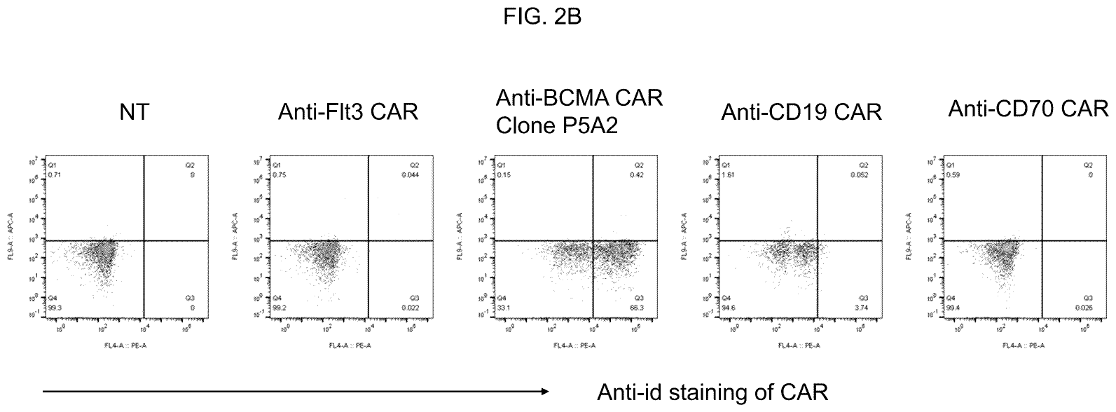

The specificity of clone H6 was tested in this experiment by flow cytometry. The data in FIG. 2A show that the anti-BCMA anti-id antibody clone H6 detected the anti-BCMA CAR clone P5A2 expressed on the cell surface. Clone H6 did not bind to the negative control anti-CD19 CAR even at a high concentration (FIG. 2A top panels). The data in FIG. 2B show that clone H6 specifically bound to anti-BCMA CAR clone P5A2 and did not bind to other CARs as negative controls.

1. An isolated antibody that specifically binds a molecule comprising an anti-BCMA antibody or an antigen-binding fragment thereof, said anti-BCMA antibody comprising the amino acid sequence of SEQ ID NO: 1 or 19.

2. The isolated antibody of claim 1, wherein the isolated antibody comprises
(a) a heavy chain variable (VH) region comprising an amino acid sequence of SEQ ID NO: 2, and a light chain variable (VL) region comprising an amino acid sequence of SEQ ID NO: 4;
(b) a VH region comprising an amino acid sequence of SEQ ID NO: 20, and a VL region comprising an amino acid sequence of SEQ ID NO: 22; or
(c) a VH region comprising an amino acid sequence of SEQ ID NO: 33, and a VL region comprising an amino acid sequence of SEQ ID NO: 35.

3. The isolated antibody of claim 1, wherein the isolated antibody comprises a VH region that comprises
(a) a heavy chain CDR1 comprising an amino acid sequence of SEQ ID NO: 6, 7 or 8, a heavy chain CDR2 comprising an amino acid sequence of SEQ ID NO: 9 or 10, and a heavy chain CDR3 comprising an amino acid sequence of SEQ ID NO: 11;
(b) a heavy chain CDR1 comprising an amino acid sequence of SEQ ID NO: 24, 25 or 26, a heavy chain CDR2 comprising an amino acid sequence of SEQ ID NO: 27 or 28, and a heavy chain CDR3 comprising an amino acid sequence of SEQ ID NO: 29; or
(c) a heavy chain CDR1 comprising an amino acid sequence of SEQ ID NO: 37, 38 or 39, a heavy chain CDR2 comprising an amino acid sequence of SEQ ID NO: 40 or 41, and a heavy chain CDR3 comprising an amino acid sequence of SEQ ID NO: 42.

4. The isolated antibody of claim 1, wherein the isolated antibody further comprises a VL region that comprises
(a) a light chain CDR1 comprising an amino acid sequence of SEQ ID NO: 12, a light chain CDR2 comprising an amino acid sequence of SEQ ID NO: 13, and a light chain CDR3 comprising an amino acid sequence of SEQ ID NO: 14;
(b) a light chain CDR1 comprising an amino acid sequence of SEQ ID NO: 30, a light chain CDR2 comprising an amino acid sequence of SEQ ID NO: 31, and a light chain CDR3 comprising an amino acid sequence of SEQ ID NO: 32; or
(c) a light chain CDR1 comprising an amino acid sequence of SEQ ID NO: 43, a light chain CDR2 comprising an amino acid sequence of SEQ ID NO: 44, and a light chain CDR3 comprising an amino acid sequence of SEQ ID NO: 45.

5. The isolated antibody of claim 4, wherein the VH region comprises an amino acid sequence that is at least 95% identical to SEQ ID NO: 2, and a VL region comprises an amino acid sequence that is at least 95% identical to SEQ ID NO: 4

6. The isolated antibody of claim 5, wherein the VH region comprises an amino acid sequence of SEQ ID NO: 2, and a VL region comprises an amino acid sequence of SEQ ID NO: 4.

7. The isolated antibody of claim 4, wherein the VH region comprises an amino acid sequence that is at least 95% identical to SEQ ID NO: 20, and a VL region comprises an amino acid sequence that is at least 95% identical to SEQ ID NO: 22.

8. The isolated antibody of claim 7, wherein the VH region comprises an amino acid sequence of SEQ ID NO: 20, and a VL region comprises an amino acid sequence of SEQ ID NO: 22.

9. The isolated antibody of claim 4, wherein the VH region comprises an amino acid sequence that is at least 95% identical to SEQ ID NO: 33, and a VL region comprises an amino acid sequence that is at least 95% identical to SEQ ID NO: 35.

10. The isolated antibody of claim 9, wherein the VH region comprises an amino acid sequence of SEQ ID NO: 33, and a VL region comprises an amino acid sequence of SEQ ID NO: 35.

11. The isolated antibody of claim 1,
wherein the isolated antibody comprises a heavy chain variable region comprising VH CDR1, VH CDR2, and VH CDR3 and a light chain variable region comprising VL CDR1, VL CDR2 and VL CDR3, wherein the VH CDR1 comprises the amino acid sequence selected from the group consisting of SEQ ID NO: 6, SEQ ID NO: 7, and SEQ ID NO: 8, the VH CDR2 comprises the amino acid sequence selected from the group consisting of SEQ ID NO: 9 and SEQ ID NO: 10, and the VH CDR3 comprises the amino acid sequence SEQ ID NO: 11, and wherein the VL CDR1 comprises the amino acid sequence of SEQ ID NO:12, the VL CDR2 comprises the amino acid sequence of SEQ ID NO:13, and the VL CDR3 comprises the amino acid sequence of SEQ ID NO: 14;
wherein the isolated antibody comprises a heavy chain variable region comprising VH CDR1, VH CDR2, and VH CDR3 and a light chain variable region comprising VL CDR1, VL CDR2 and VL CDR3, wherein the VH CDR1 comprises the amino acid sequence selected from the group consisting of SEQ ID NO: 24, SEQ ID NO: 25, and SEQ ID NO: 26, the VH CDR2 comprises the amino acid sequence selected from the group consisting of SEQ ID NO: 27 and SEQ ID NO: 28, and the VH CDR3 comprises the amino acid sequence SEQ ID NO: 29, and wherein the VL CDR1 comprises the amino acid sequence of SEQ ID NO:30, the VL CDR2 comprises the amino acid sequence of SEQ ID NO: 31, and the VL CDR3 comprises the amino acid sequence of SEQ ID NO: 32; or
wherein the isolated antibody comprises a heavy chain variable region comprising VH CDR1, VH CDR2, and VH CDR3 and a light chain variable region comprising VL CDR1, VL CDR2 and VL CDR3, wherein the VH CDR1 comprises the amino acid sequence selected from the group consisting of SEQ ID NO: 37, SEQ ID NO: 38, and SEQ ID NO: 39, the VH CDR2 comprises the amino acid sequence selected from the group consisting of SEQ ID NO: 40 and SEQ ID NO: 41, and the VH CDR3 comprises the amino acid sequence SEQ ID NO: 42, and wherein the VL CDR1 comprises the amino acid sequence of SEQ ID NO: 43, the VL CDR2 comprises the amino acid sequence of SEQ ID NO: 44, and the VL CDR3 comprises the amino acid sequence of SEQ ID NO: 45.

12. (canceled)

13. (canceled)

14. (canceled)

15. (canceled)

16. (canceled)

17. A polynucleotide encoding the isolated antibody of claim 1.

18. A vector comprising the polynucleotide of claim 17.

19. A cell comprising the polynucleotide of claim 17.

20. A method of making an isolated antibody that specifically binds a molecule comprising an anti-BCMA scFv comprising the amino acid sequence of SEQ ID NO: 1 or 19, the method comprising growing or culturing the cell of claim 19 under suitable conditions.

21. A method of determining a number of cells expressing an anti-BCMA antibody comprising amino acid sequences of SEQ ID NOs: 15 and 16 or SEQ ID NOs: 17 and 18, comprising contacting the cells with the isolated antibody of claim 1 and determining the number of cells expressing the anti-BCMA antibody in the cells.

22. A method of determining a presence or absence of cells expressing an anti-BCMA antibody comprising amino acid sequences of SEQ ID NOs: 15 and 16 or SEQ ID NOs: 17 and 18, comprising contacting the cells with the isolated antibody of claim 1 and determining the presence or absence of cells expressing the anti-BCMA antibody in the cells.

23. (canceled)

24. (canceled)

25. (canceled)

26. A method of selecting cells that express an anti-BCMA CAR comprising an amino acid sequence of SEQ ID NO: 1 or 19, comprising
(a) contacting the cells with an isolated antibody of claim 1; and
(b) selecting the cells bound with the antibody,
wherein the isolated antibody further comprises a detectable label.

27. (canceled)

28. (canceled)

29. A method of purifying an anti-BCMA antibody, or antigen-binding fragment thereof, wherein the anti-BMCA antibody comprises amino acid sequences of SEQ ID NOs: 15 and 16 or SEQ ID NOs: 17 and 18:
(a) contacting a sample comprising the anti-BCMA antibody with the isolated antibody of claim 1 to form a complex; and
(b) purifying the complex comprising the anti-BCMA antibody and the isolated antibody from the sample.

30. A method of stimulating cells that express an anti-BCMA chimeric antigen receptor (anti-BCMA CAR T cells), the method comprising incubating the anti-BCMA CAR T cells with the isolated antibody of claim 1, wherein the anti-BCMA CAR T cells express the anti-BCMA antibody comprising amino acid sequences of SEQ ID NOs:15 and 16 or SEQ ID NOs:17 and 18.
